(19) United States
(12) Patent Application Publication
(10) Pub. No.: US 2022/0364095 A1
(43) Pub. Date: Nov. 17, 2022

(54) TUNABLE ANCHOR FOR LIPOSOMAL SPHERICAL NUCLEIC ACID ASSEMBLY

(71) Applicant: NORTHWESTERN UNIVERSITY, Evanston, IL (US)

(72) Inventors: Chad A. Mirkin, Wilmette, IL (US); Jasper Wilson Dittmar, Evanston, IL (US)

(21) Appl. No.: 17/743,422

(22) Filed: May 12, 2022

Related U.S. Application Data
(60) Provisional application No. 63/187,786, filed on May 12, 2021.

(51) Int. Cl. C12N 15/117; A61K 39/39

(52) U.S. Cl. CPC C12N 15/117; A61K 39/39; A61K 2039/55505

(57) ABSTRACT

The disclosure is generally related to spherical nucleic acids (SNAs), structures comprising a nanoparticle core surrounded by a shell of oligonucleotides. In some aspects the disclosure provides a spherical nucleic acid (SNA) comprising: (a) a nanoparticle core; and (b) a shell of oligonucleotides attached to the nanoparticle core, wherein one or more oligonucleotides in the shell of oligonucleotides is attached to the nanoparticle core through a hydrophobic anchor comprising one or more dodecane (C12) subunits. Methods of making and using the SNAs are also provided herein.

### CROSS-REFERENCE TO RELATED APPLICATIONS

This application claims the priority benefit under 35 U.S.C. § 119(e) of U.S. Provisional Patent Application No. 63/187,786, filed May 12, 2021, which is incorporated herein by reference in its entirety.

### STATEMENT OF GOVERNMENT SUPPORT

This invention was made with government support under grant number CA199091 awarded by the National Institutes of Health and grant number N00014-16-1-3117 awarded by The Office of Naval Research. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2021-112_Seqlisting.txt”, which was created on May 12, 2022 and is 991 bytes in size. The subject matter of the Sequence Listing is incorporated herein in its entirety by reference.

### BACKGROUND

Liposomes have proven to be one of the most versatile and effective platforms for drug delivery due to their biocompatibility, unique biodistribution profiles, and the ability to encapsulate and protect a variety of therapeutic cargo [Malam, Y., Loizidou, M. & Seifalian, A. M. Liposomes and nanoparticles: nanosized vehicles for drug delivery in cancer. Trends in pharmacological sciences 30, 592-599, doi:10.1016/j.tips.2009.08.004 (2009); Bulbake, U., Doppalapudi, S., Kommineni, N. & Khan, W. Liposomal Formulations in Clinical Use: An Updated Review. Pharmaceutics 9, 12, doi:10.3390/pharmaceutics9020012 (2017); Adlakha-Hutcheon, G., Bally, M. B., Shew, C. R. & Madden, T. D. Controlled destabilization of a liposomal drug delivery system enhances mitoxantrone antitumor activity. Nat Biotechnol 17, 775-779, doi:10.1038/11710 (1999)]. The translation of liposomal drugs from the benchtop to the clinic has been driven by the development of surface modifications which improve stability and bioavailability [Bulbake, U., Doppalapudi, S., Kommineni, N. & Khan, W. Liposomal Formulations in Clinical Use: An Updated Review. Pharmaceutics 9, 12, doi:10.3390/pharmaceutics9020012 (2017); Immordino, M. L., Dosio, F. & Cattel, L. Stealth liposomes: review of the basic science, rationale, and clinical applications, existing and potential. Int J Nanomedicine 1, 297-315 (2006); Romberg, B., Hennink, W. E. & Storm, G. in Pharm Res Vol. 25 55-71 (2008)]. Densely modifying the surface of a liposome with nucleic acids to create the spherical nucleic acid (SNA) can improve the stability of liposomal cargo [Banga, R. J., Chernyak, N., Narayan, S. P., Nguyen, S. T. & Mirkin, C. A. Liposomal spherical nucleic acids. J Am Chem Soc 136, 9866-9869, doi:10.1021/ja504845f (2014)]. . Efforts to improve liposomal SNA stability have been made by modifying the lipophilic anchor which tethers the oligonucleotide to the liposome [Meckes, B., Banga, R. J., Nguyen, S. T. & Mirkin, C. A. Enhancing the Stability and Immunomodulatory Activity of Liposomal Spherical Nucleic Acids through Lipid-Tail DNA Modifications. Small 14, doi:UNSP 1702909 10.1002/sm11.201702909 (2018)].

### SUMMARY

Spherical Nucleic Acids (SNAs) are a nanoscale architecture comprised of a nanoparticle core (e.g., liposome) with a dense radial surface arrangement of oligonucleotides (e.g., immunostimulatory CpG motif containing DNA). These constructs have shown tremendous immunotherapeutic potential due to their rapid uptake by antigen-presenting cells (APCs), prolonged serum half-lives, and superior antitumor immune responses compared to commonly employed simple mixtures of immunostimulatory DNA and antigenic peptides (1, 2). Indeed, SNAs are currently in numerous clinical trials (3). The stability of the oligonucleotide (e.g., DNA) attachment to the surface of the nanoparticle core (e.g., liposome) is a critical parameter, as it defines the longevity and strength of their biological interactions. The present disclosure provides a modular anchor design to precisely tune the strength of the oligonucleotide (e.g., DNA) attachment to the core, achieving high stabilities, and also discloses the relationship between SNA stability and immunostimulation.

Immunotherapy targeted towards various diseases (e.g., cancer) is a quickly growing and developing field of medicine. Liposomal materials have also been FDA approved and are biocompatible, easing the transition of the materials described herein to the market. The technology described herein provides improvements in both immunotherapy and liposomal technologies.

Applications of the technology described herein include, but are not limited to:
Immunotherapy
Drug delivery
Control over dissociation rates

Advantages of the technology described herein include, but are not limited to:
Increased oligonucleotide loading of spherical nucleic acids (SNAs), e.g., liposomal spherical nucleic acids (LSNAs)
Higher LSNA serum stability
Fine tuning of oligonucleotide dissociation rates
Increased immunostimulation
Greater antigen-specific immune responses

Accordingly, in some aspects the present disclosure provides a spherical nucleic acid (SNA) comprising: (a) a nanoparticle core; and (b) a shell of oligonucleotides attached to the nanoparticle core, wherein one or more oligonucleotides in the shell of oligonucleotides is attached to the nanoparticle core through a hydrophobic anchor comprising one or more dodecane (C12) subunits. In some embodiments, the hydrophobic anchor comprises 1 to about 20 dodecane subunits. In some embodiments, the hydrophobic anchor comprises 9 dodecane subunits. In further embodiments, the SNA comprises an antigen, wherein the antigen is encapsulated in the nanoparticle core, attached to one or more oligonucleotides in the shell of oligonucleotides through a linker, attached to the external surface of the nanoparticle core through a linker, or a combination thereof. In some embodiments, the antigen is attached through the linker to each oligonucleotide in the shell of oligonucleotides. In further embodiments, the antigen is attached through the linker to an oligonucleotide that is hybridized to an oligonucleotide in the shell of oligonucleotides that is attached to the nanoparticle core. In various embodiments, the shell of oligonucleotides comprises an immunostimulatory oligonucleotide, a targeting oligonucleotide, an inhibitory oligonucleotide, a non-targeting oligonucleotide, or a combination thereof. In some embodiments, the immunostimulatory oligonucleotide is a toll-like receptor (TLR) agonist. In further embodiments, each oligonucleotide in the shell of oligonucleotides is a toll-like receptor (TLR) agonist. In still further embodiments, the TLR is toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), toll-like receptor 13 (TLR13), or a combination thereof. In some embodiments, the TLR is TLR9. In further embodiments, the immunostimulatory oligonucleotide comprises a CpG nucleotide sequence. In further embodiments, one or more oligonucleotides in the shell of oligonucleotides comprises or consists of the sequence of 5′-TCCATGACGTTCCTGACGTT-3′ (CpG 1826; SEQ ID NO: 3). In still further embodiments, one or more oligonucleotides in the shell of oligonucleotides comprises or consists of the sequence of 5′-TCGTCGTTTTGTCGTTTTGTCGTT-3′ (CpG 7909; SEQ ID NO: 4). In various embodiments, at least about 10%, 20%, 30%, 40%, 50%, 60%, 70% 80%, 90%, 95%, or 100% of oligonucleotides in the shell of oligonucleotides is an immunostimulatory oligonucleotide. In various embodiments, the linker is a carbamate alkylene disulfide linker, a thiol linker, a disulfide linker, an amide alkylene disulfide linker, an amide alkylene thio-succinimidyl linker, or a combination thereof. In various embodiments, the nanoparticle core is a micellar core, a dendrimer core, a liposomal core, a lipid nanoparticle core, or a combination thereof. In some embodiments, the nanoparticle core is a liposome. In further embodiments, the liposome comprises 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cholesterol, or a combination thereof. In some embodiments, the liposome comprises 1,2-dipalmitoyl-sn-glycero-3-phosphocholine (DPPC). In some embodiments, each oligonucleotide in the shell of oligonucleotides comprises the hydrophobic anchor. In further embodiments, the hydrophobic anchor is attached to the 5′ end or the 3′ end of the one or more oligonucleotides. In various embodiments, the shell of oligonucleotides comprises DNA oligonucleotides, RNA oligonucleotides, or a combination thereof. In further embodiments, the shell of oligonucleotides comprises DNA oligonucleotides and RNA oligonucleotides. In various embodiments, the shell of oligonucleotides comprises single-stranded DNA, double-stranded DNA, single-stranded RNA, double-stranded RNA, or a combination thereof. In some embodiments, one or more oligonucleotides in the shell of oligonucleotides is a modified oligonucleotide. In various embodiments, the shell of oligonucleotides comprises about 2 to about 500 oligonucleotides. In some embodiments, the shell of oligonucleotides comprises about 75 oligonucleotides. In various embodiments, the shell of oligonucleotides is about 5 to about 1000 nucleotides in length. In further embodiments, each oligonucleotide in the shell of oligonucleotides is about 10 to about 50 nucleotides in length. In various embodiments, diameter of the SNA is about 1 nanometer (nm) to about 500 nm. In further embodiments, diameter of the SNA is less than or equal to about 80 nanometers. In still further embodiments, diameter of the SNA is less than or equal to about 50 nanometers. In various embodiments, the inhibitory oligonucleotide is an antisense oligonucleotide, small interfering RNA (siRNA), an aptamer, a short hairpin RNA (shRNA), a DNAzyme, or an aptazyme. In various embodiments, the antigen is a tumor antigen, a viral antigen, a bacterial antigen, or a combination thereof.

In some aspects, the disclosure provides a composition comprising a plurality of the SNA of the disclosure. In some embodiments, at least two SNAs in the plurality comprise a different nanoparticle core.

In further aspects, the disclosure provides a pharmaceutical formulation comprising a plurality of the SNA of the disclosure, or a composition of the disclosure, and a pharmaceutically acceptable carrier or diluent.

In some aspects, the disclosure provides a vaccine comprising a SNA, composition, or pharmaceutical formulation of the disclosure. In some embodiments, the vaccine comprises an adjuvant.

In some aspects, the disclosure provides an antigenic composition comprising a SNA of the disclosure in a pharmaceutically acceptable carrier, diluent, stabilizer, or preservative, or a pharmaceutical formulation of the disclosure, wherein the antigenic composition is capable of generating an immune response including dendritic cell activation, antibody generation, cytotoxic T cell activation, helper T cell activation, or a protective immune response in a subject. In some embodiments, the immune response includes an antibody response. In some embodiments, the antibody response is a neutralizing antibody response or a protective antibody response.

In some aspects, the disclosure provides a method of inhibiting expression of a gene product comprising hybridizing a polynucleotide encoding the gene product to an inhibitory oligonucleotide as described herein, wherein hybridizing between the polynucleotide and the inhibitory oligonucleotide occurs over a length of the polynucleotide with a degree of complementarity sufficient to inhibit expression of the gene product. In some embodiments, expression of the gene product is inhibited in vivo or in vitro.

In some aspects, the disclosure provides a method of producing an immune response in a subject, comprising administering to the subject an effective amount of a SNA, composition, pharmaceutical formulation, vaccine, or antigenic composition of the disclosure, thereby producing an immune response in the subject. In some embodiments, the immune response includes an antibody response. In some embodiments, the antibody response is a total antigen-specific antibody response. In some embodiments, the antibody response is a neutralizing antibody response or a protective antibody response.

In further aspects, the disclosure provides a method of immunizing a subject against one or more antigens comprising administering to the subject an effective amount of a SNA, composition, pharmaceutical formulation, vaccine, or antigenic composition of the disclosure, thereby immunizing the subject against the one or more antigens. In some embodiments, the composition or the vaccine is a cancer vaccine.

In some aspects, the disclosure provides a method of treating a disorder comprising administering to a subject an effective amount of a SNA, composition, pharmaceutical formulation, vaccine, or antigenic composition of the disclosure, thereby treating the disorder in the subject. In some embodiments, the disorder is a cancer or an infection. In further embodiments, the infection is a viral infection or a bacterial infection. In still further embodiments, the cancer is bladder cancer, breast cancer, cervical cancer, colon cancer, rectal cancer, endometrial cancer, glioblastoma, kidney cancer, leukemia, liver cancer, lung cancer, melanoma, lymphoma, non-Hodgkin lymphoma, osteocarcinoma, ovarian cancer, pancreatic cancer, prostate cancer, thyroid cancer, and human papilloma virus-induced cancer, or a combination thereof. In some embodiments, methods of the disclosure further comprise administering a therapeutic agent. In further embodiments, the therapeutic agent is an anti-programmed cell death protein 1 (PD-1) antibody, an anti-programmed death-ligand 1 (PD-L1) antibody, a cytotoxic T lymphocyte antigen 4 (CTLA-4) antibody, or a combination thereof. In some embodiments, the administering results in a less than 25% increase in serum level of a cytokine in the subject compared to serum level of the cytokine in a subject that was not administered the SNA. In further embodiments, the cytokine is interleukin-2 (IL-2), interleukin-6 (IL-6), interleukin-10 (IL-10), C-X-C Motif Chemokine Ligand 1 (CXCL1), tumor necrosis factor a (TNF-α), interleukin-1 (IL-1), interferon, or a combination thereof.

### BRIEF DESCRIPTION OF THE FIGURES

FIG. 1 depicts a cartoon of an immunostimulatory SNA and the structure of the anchor which can be oligomerized to tune LSNA stability (left panel). The upper right panel shows a cartoon of a LSNA serum dissociation experiment and the lower right panel shows dissociation data with half-lives derived from a single exponential decay fit of the data. 9 C12 spacers resulted in the most stable LSNA which is also more stable than previous formulations containing a cholesterol or lipid on the 3′ terminus of the oligonucleotide.

FIG. 2 shows that the loading of LSNAs increased with a more hydrophobic anchor comprised of 9 C12 spacers (400 oligos per 50 nm liposome, 1.75 pmol/cm²) compared to 4 C12 spacers (75 oligos per liposome, 0.33 pmol/cm²).

FIG. 3 shows that LSNA uptake and activation of bone-marrow derived dendritic cell was greatest for LSNAs with an anchor comprising 9 C12 spacers. Statistical significance was calculated by one-way ANOVA with multiple comparisons using the bon-ferroni correction.

FIG. 4 shows the functional relationship between LSNA uptake and activation utilizing an anchor as described herein to tune stability.

FIG. 5A shows that LSNAs formulated with MHCI peptides promoted stronger immune responses in vivo. Vaccination of mice with LSNAs utilizing the (C12)₉ anchor and SARS-COV-2 peptide (left) or prostate specific membrane antigen (PSMA₆₃₄, middle) resulted in greater antigen specific interferon gamma (IFN-γ) secretion by CD8⁺ splenocytes and promoted memory T cell formation (right). FIG. 5B shows that vaccination of AAD mice (3-4) with 9C12 SNAs containing a peptide derived from SARS-COV-2 spike receptor binding domain and affinity for human HLA-A*02.01 initiated a more robust CD8 T cell response towards the peptide compared to either linear CpG and the peptide or a cholesterol-anchored SNA. Flow cytometry of CD8+ splenocytes (left) as well as ELISpot (right) both showed increased IFN-γ secretion for T cells raised from (C12)₉ SNA vaccination after incubation with the peptide. One-way ANOVA with Tukey's correction was used to determine statistical significance.

FIG. 6 shows a) SNA schematic and chemical structure of the C12 anchor. b) Uptake of Cy5-labeled SNAs, CD80 expression by BMDCs, and SNA half-lives in 10% fetal bovine serum as a function of anchor. c) Killing of E.G7-OVA lymphoma cells by CD8⁺ T cells from C57BL/6 mice immunized with SNAs containing the OVA1 peptide. Error bars are 95% CI of the mean.

FIG. 7 shows that SNA adjuvants reduce the risk of acute cytokine release. Proinflammatory serum cytokine concentrations after subcutaneous administration of 6 nmol CpG oligonucleotide (n=4). All significant differences in cytokine concentrations are presented via ANOVA with Tukey-corrected comparisons. Error bars represent s.d. *p<0.05, **p<0.01, ***p<0.001, ****p<0.0001.

FIG. 8 shows A+B) Dose-dependent activation of BMDCs with Au SNAs and (C12)₉ SNAs with varied DNA shell density. C) Activation of BMDCs with 200 nmol (1 μM) CpG as a function of both anchor and liposome core chemistries. D) Dose-dependent activation of human antigen-presenting cells. For all panels, statistically significant comparisons to (C12)₉ SNAs from the results of ANOVA with Tukey-corrected comparisons with a representative example of 3 independent experiments is shown. Error bars represent s.d. *p<0.05, **p<0.01, ***p<0.001, ****p<0.0001.

FIG. 9 demonstrates that (C12)₉ SNAs elicited potent antigen-specific CD8 T Cell immunity and inhibit tumor growth. A) Injection timeline for immunizations. B) Percentage of raised CD8⁺ splenic T cells which recognize OVA1 antigen after vaccination (n=7, from two experiments) is increased by. C) Percentage of polyfunctional (CD107a⁺IFN-γ⁺) splenic CD8 T Cells (n=7, from two independent experiments). D) IFN-γ spot-forming cells (SFCs) after stimulation of splenocytes with OVA1 peptide (n=4 biological replicates) E) Representative counted ELISpot images. F) Dosing schedule for treatment of E.G7-OVA lymphoma. G) E.G7-OVA tumor growth plots with different cancer vaccine treatments (n=10-12 mice from three independent blinded experiments). H) Kaplan-Meier survival curves of E.G7-OVA lymphoma with statistical significance assessed via the Mantel-Cox log-rank test. I) Surviving mice are resistant to rechallenge with lymphoma cells. All error bars represent s.d. Statistical significance in B, C, D, G, and I was determined by one-way ANOVA with Tukey corrected comparisons. *p<0.05, **p<0.01, ***p<0.001 ****, p<0.0001

### DETAILED DESCRIPTION

In some aspects, the disclosure is directed to SNAs comprising a liposomal core and a dense radial arrangement of oligonucleotides on the surface of the liposomal core. Liposomes have been broadly used to alter drug properties. By encapsulating cargo in the core, they are able to increase accumulation in different organs, protect cargo from degradation, and deliver their cargo to cells. However, the broad application of liposomes has been limited for drug delivery applications mainly by their poor stability in biological environments. The present disclosure provides, in various aspects, strategies to overcome the relatively poor stability of liposomes through iterative addition of hydrophobic units to an oligonucleotide to generate liposomal spherical nucleic acids with enhanced and tunable stability. The present disclosure addresses the poor stability of liposomal based constructs, allowing the development of more potent liposomal based drugs which utilize less material. Promoting strong antigen-specific immune responses has also been an outstanding challenge in the field of immunotherapy, which the present disclosure addresses through providing a platform to generate stronger CD8 T Cell immune responses than conventional LSNA therapies which contain a cholesterol anchor.

In various aspects, the disclosure provides methods of synthesizing liposomal spherical nucleic acids (LSNAs) using an anchor comprising oligomerized hydrophobic units, allowing fine tuning and improvement of LSNA stability.

As used in this specification and the appended claims, the singular forms “a,” “an,” and “the” include plural reference unless the context clearly dictates otherwise.

The terms “polynucleotide” and “oligonucleotide” are interchangeable as used herein.

A range includes each individual member. Thus, for example, a group having 1-3 members refers to groups having 1, 2, or 3 members. Similarly, a group having 6 members refers to groups having 1, 2, 3, 4, or 6 members, and so forth.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting a SNA to a subject in need of treatment with such an agent. Such modes include, but are not limited to, oral, topical, intravenous, intraarterial, intraperitoneal, intramuscular, intratumoral, intradermal, intranasal, and subcutaneous administration.

As used herein, “treating” and “treatment” refers to any reduction in the severity and/or onset of symptoms associated with a disease (e.g., cancer). Accordingly, “treating” and “treatment” includes therapeutic and prophylactic measures. One of ordinary skill in the art will appreciate that any degree of protection from, or amelioration of, the disease (e.g., cancer) is beneficial to a subject, such as a human patient. The quality of life of a patient is improved by reducing to any degree the severity of symptoms in a subject and/or delaying the appearance of symptoms.

As used herein, a “targeting oligonucleotide” is an oligonucleotide that directs a SNA to a particular tissue and/or to a particular cell type or it is an oligonucleotide that detects a target analyte. In some embodiments, a targeting oligonucleotide is an aptamer. Thus, in some embodiments, a SNA of the disclosure comprises an aptamer attached to the exterior of the nanoparticle core, wherein the aptamer is designed to bind one or more receptors on the surface of a certain cell type, or the aptamer is designed to detect a target analyte. Target analytes contemplated by the disclosure include without limitation a protein, an ion, a small molecule, a lipid, a carbohydrate, an oligosaccharide, a cell, an oligonucleotide, or a combination thereof.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence. In any of the aspects or embodiments of the disclosure, the immunostimulatory oligonucleotide is a toll-like receptor (TLR) agonist (e.g., a toll-like receptor 9 (TLR9) agonist). In various embodiments, about, less than about, or at least about 5%, 10%, 20%, 30%, 40%, 50%, 60%, 70% 80%, 90%, 95%, or 100% of oligonucleotides in the shell of oligonucleotides of a SNA as described herein are immunostimulatory oligonucleotides.

The term “inhibitory oligonucleotide” refers to an oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include, without limitation, isolated or synthetic short hairpin RNA (shRNA or DNA), an antisense oligonucleotide (e.g., antisense RNA or DNA, chimeric antisense DNA or RNA), miRNA and miRNA mimics, small interfering RNA (siRNA), DNA or RNA inhibitors of innate immune receptors, an aptamer, a DNAzyme, or an aptazyme.

The term “non-targeting oligonucleotide” refers an oligonucleotide included, in some embodiments, in the shell of oligonucleotides of a SNA that is not associated with a particular activity (e.g., an immunostimulatory activity) but instead is used to achieve a certain density of oligonucleotides on the external surface of a SNA. Non-limiting examples of non-targeting oligonucleotides are an oligonucleotide comprising a scrambled nucleotide sequence and/or a homopolymeric oligonucleotide (e.g., a polythymidine oligonucleotide (such as T20)).

An “antigenic composition” is a composition of matter suitable for administration to a human or animal subject (e.g., in an experimental or clinical setting) that is capable of eliciting a specific immune response, e.g., against an antigen, such as one or more of the antigens described herein. In the context of this disclosure, the term antigenic composition will be understood to encompass compositions that are intended for administration to a subject or population of subjects for the purpose of eliciting a protective or palliative immune response against an antigen, such as one or more of the antigens described herein.

The term “dose” as used herein refers to a measured portion of any of the SNAs of the disclosure (e.g., a SNA, antigenic composition, pharmaceutical formulation as described herein) taken by (administered to or received by) a subject at any one time.

An “immune response” is a response of a cell of the immune system, such as a B cell, T cell, or monocyte, to a stimulus, such as a SNA as described herein. An immune response can be a B cell response, which results in the production of specific antibodies, such as antigen specific neutralizing antibodies. An immune response can also be a T cell response, such as a CD4⁺ helper T cell response or a CD8⁺ cytotoxic T cell response. B cell and T cell responses are aspects of a “cellular” immune response. An immune response an also include dendritic cell activation. As described herein, an “immune response” can also be a “treatment based” response in which the immune system is being primed while actively fighting the tumor. An immune response can also be a “humoral” immune response, which is mediated by antibodies. In some cases, the response is specific for a particular antigen (that is, an “antigen-specific response”). A “protective immune response” is an immune response that inhibits a detrimental function or activity of an antigen, or decreases symptoms (including death) that result from the antigen. Protective in this context does not necessarily require that the subject is completely protected against infection. A protective response is achieved when the subject is protected from developing symptoms of disease, or when the subject experiences a lower severity of symptoms of disease. A protective immune response can be measured, for example, by immune assays using a serum sample from an immunized subject and testing the ability of serum antibodies for inhibition of pseudoviral binding, such as: pseudovirus neutralization assay (or surrogate virus neutralization test), ELISA-neutralization assay, antibody dependent cell-mediated cytotoxicity assay (ADCC), complement-dependent cytotoxicity (CDC), antibody dependent cell-mediated phagocytosis (ADCP), enzyme-linked immunospot (ELISpot). In addition, vaccine efficacy can be tested by measuring B or T cell activation after immunization, using flow cytometry (FACS) analysis or ELISpot assay. The protective immune response can be tested by measuring resistance to antigen challenge in vivo in an animal model. In humans, a protective immune response can be demonstrated in a population study, comparing measurements of symptoms, morbidity, mortality, etc. in treated subjects compared to untreated controls. Exposure of a subject to an immunogenic stimulus, such as a SNA as described herein, elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering, e.g., an antigenic composition of the disclosure increases the magnitude of an antigen-specific response, (e.g., by increasing the breadth of produced antibodies (i.e., in the case of administering a booster that primes the immune system against a variant), by increasing antibody titer and/or affinity, by increasing the frequency of antigen specific B or T cells, by inducing maturation effector function, or a combination thereof). The “maturity and memory” of B and T cells may also be measured as an indicator of an immune response.

“Adjuvant” refers to a substance which, when added to a composition comprising an antigen, nonspecifically enhances or potentiates an immune response to the antigen in the recipient upon exposure. In any of the aspects or embodiments of the disclosure, the SNAs provided herein comprise immunostimulatory oligonucleotides (for example and without limitation, a toll-like receptor (TLR) agonist) as adjuvants. In some embodiments, a SNA of the disclosure comprises one or more antigens as described herein. Additional adjuvants contemplated for use according to the disclosure include aluminum (e.g., aluminum hydroxide), lipid-based adjuvant ASO1B, alum, MF59, in addition to TLR agonists as described herein (e.g., CpG DNA, TLR7's imiquimod, TLR8's Motolimod, TLR4's MPLA4, TLR3's Poly (I:C), or a combination thereof).

An “effective amount” or a “sufficient amount” of a substance is that amount necessary to effect beneficial or desired results, including clinical results, and, as such, an “effective amount” depends upon the context in which it is being applied. In the context of administering a SNA of the disclosure, in some embodiments an effective amount is an amount sufficient to generate an antigen specific immune response. An effective amount can be administered in one or more doses as described further herein. Efficacy can be shown in an experimental or clinical trial, for example, by comparing results achieved with a substance of interest compared to an experimental control.

All references, patents, and patent applications disclosed herein are incorporated by reference with respect to the subject matter for which each is cited, which in some cases may encompass the entirety of the document.

### Spherical Nucleic Acids (SNAs)

SNAs comprise a nanoparticle core surrounded by a shell of oligonucleotides. In various embodiments, an oligonucleotide shell is formed when at least 10% of the available surface area of the nanoparticle core (e.g., the exterior surface) includes an oligonucleotide. In further embodiments at least 20%, 30%, 40%, 50%, 60%, 70%, 80%, 90%, 95%, 96%, 97%, 98% or 99% of the available surface area of the nanoparticle core includes an oligonucleotide. The oligonucleotides of the oligonucleotide shell may be oriented in a variety of directions. In some embodiments the oligonucleotides are oriented radially outwards. The oligonucleotide shell comprises one or more oligonucleotides attached to the nanoparticle core. The spherical architecture of the oligonucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis (see, e.g., U.S. Patent Application Publication No. 2010/0233270, incorporated by reference herein in its entirety).

Thus, in any of the aspects or embodiments of the disclosure, a spherical nucleic acid (SNA) is provided comprising: (a) a nanoparticle core; and (b) a shell of oligonucleotides attached to the nanoparticle core, wherein one or more oligonucleotides in the shell of oligonucleotides is attached to the nanoparticle core through a hydrophobic anchor comprising one or more dodecane (C12) subunits. In any of the aspects or embodiments of the disclosure, the shell of oligonucleotides is attached to the external surface of the nanoparticle core.

In any of the aspects or embodiments of the disclosure, the nanoparticle core is a lipid-based core. In various embodiments, the nanoparticle core is a micellar core, a dendrimer core, a liposomal core, a lipid nanoparticle core, or a combination thereof.

Liposomes are spherical, self-closed structures in a varying size range comprising one or several hydrophobic lipid bilayers with a hydrophilic core. The diameter of these lipid based carriers range from 0.15-1 micrometers, which is significantly higher than an effective therapeutic range of 20-100 nanometers. Liposomes termed small unilamellar vesicles (SUVs), can be synthesized in the 20-50 nanometer size range, but encounter challenges such as instability and aggregation leading to inter-particle fusion. This inter-particle fusion limits the use of SUVs in therapeutics. In some aspects, the disclosure provides liposomal spherical nucleic acids (LSNAs) comprising a liposomal core and a shell of oligonucleotides attached to the liposomal core, wherein one or more oligonucleotides in the shell of oligonucleotides is attached to the nanoparticle core through a hydrophobic anchor comprising one or more dodecane (C12) subunits.

Liposomal particles, for example as disclosed in International Patent Application No. PCT/US2014/068429 (incorporated by reference herein in its entirety) are therefore provided by the disclosure. Liposomal particles of the disclosure have at least a substantially spherical geometry, an internal side and an external side, and comprise a plurality of lipid groups. In various embodiments, the plurality of lipid groups comprises a lipid selected from the group consisting of the phosphatidylcholine, phosphatidylglycerol, and phosphatidylethanolamine families of lipids. Lipids contemplated by the disclosure include, without limitation, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), 1,2-dioleoyl-sn-glycero-3-phospho-(1′-rac-glycerol) (DOPG), 1,2-distearoyl-sn-glycero-3-phosphoethanolamine (DSPE), cardiolipin, lipid A, monophosphoryl Lipid A (MPLA), or a combination thereof. In some embodiments, the lipid is 1,2-dipalmitoyl-sn-glycero-3-phosphocholine (DPPC). In some aspects the disclosure provides a spherical nucleic acid (SNA) comprising: (a) a nanoparticle core, wherein the nanoparticle core is a liposome comprising 1,2-dipalmitoyl-sn-glycero-3-phosphocholine (DPPC); and (b) a shell of oligonucleotides attached to the nanoparticle core, wherein one or more or all oligonucleotides in the shell of oligonucleotides is attached to the nanoparticle core through a hydrophobic anchor comprising nine dodecane (C12) subunits.

As described herein, the disclosure provides SNAs comprising (a) a nanoparticle core; and (b) a shell of oligonucleotides attached to the nanoparticle core, wherein one or more oligonucleotides in the shell of oligonucleotides is attached to the nanoparticle core through a hydrophobic anchor comprising one or more dodecane (C12) subunits. In some embodiments, each oligonucleotide in the shell of oligonucleotides comprises the hydrophobic anchor. In various embodiments, the hydrophobic anchor is attached to the 5′ end or the 3′ end of the one or more oligonucleotides. In some embodiments, at least one oligonucleotide in the shell of oligonucleotides is attached to the exterior of the liposomal core through a lipid anchor group. In various embodiments, the lipid anchor group is attached to the 5′ end or the 3′ end of the at least one oligonucleotide. In still further embodiments, the lipid anchor group is tocopherol, palmitoyl, dipalmitoyl, stearyl, distearyl, or cholesterol. Thus, in various embodiments, at least one of the oligonucleotides in the shell of oligonucleotides is an oligonucleotide-lipid conjugate containing a lipid anchor group, wherein said lipid anchor group is adsorbed into the lipid bilayer. Methods of making oligonucleotides comprising a lipid anchor are known in the art (see, e.g., U.S. Pat. No. 10,792,251, incorporated by reference herein in its entirety).

Methods of making a liposomal SNA (LSNA) are described herein and are generally known (see, e.g., Wang et al., Proc. Natl. Acad. Sci. 2019, 116 (21), 10473-10481, incorporated by reference herein in its entirety).

Lipid nanoparticle spherical nucleic acids (LNP-SNAs) are comprised of a lipid nanoparticle core decorated with a shell of oligonucleotides. The spherical architecture of the oligonucleotide shell confers unique advantages over traditional nucleic acid delivery methods, including entry into nearly all cells independent of transfection agents, resistance to nuclease degradation, sequence-based function, targeting, and diagnostics. The lipid nanoparticle core comprises an ionizable lipid, a phospholipid, a sterol, and a lipid-polyethylene glycol (lipid-PEG) conjugate. In some aspects, the disclosure provides lipid nanoparticle spherical nucleic acids (LNP-SNAs) comprising a lipid nanoparticle core and a shell of oligonucleotides attached to the lipid nanoparticle core, wherein one or more oligonucleotides in the shell of oligonucleotides is attached to the nanoparticle core through a hydrophobic anchor comprising one or more dodecane (C12) subunits.

In some embodiments, the ionizable lipid is dilinoleylmethyl-4-dimethylaminobutyrate (DLin-MC3-DMA), 2,2-Dilinoleyl-4-dimethylaminoethyl-[1,3]-dioxolane (DLin-KC2-DMA), C12-200, 1,2-dioleoyl-3-dimethylammonium-propane (DODAP), similar lipid/lipidoid structures, or a combination thereof. In some embodiments, the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), monophosphoryl Lipid A (MPLA), or a combination thereof. In further embodiments, the sterol is 3β-Hydroxycholest-5-ene (Cholesterol), 9,10-Secocholesta-5,7,10(19)-trien-36-ol (Vitamin D3), 9,10-Secoergosta-5,7,10(19),22-tetraen-36-ol (Vitamin D2), Calcipotriol, 24-Ethyl-5,22-cholestadien-3β-ol (Stigmasterol), 22,23-Dihydrostigmasterol (β-Sitosterol), 3,28-Dihydroxy-lupeol (Betulin), Lupeol, Ursolic acid, Oleanolic acid, 24α-Methylcholesterol (Campesterol), 24-Ethylcholesta-5,24(28)E-dien-3β-ol (Fucosterol), 24-Methylcholesta-5,22-dien-3β-ol (Brassicasterol), 24-Methylcholesta-5,7,22-trien-3β-ol (Ergosterol), 9,11-Dehydroergosterol, Daucosterol, or any of the foregoing sterols modified with one or more amino acids. In some embodiments, the lipid-polyethylene glycol (lipid-PEG) conjugate comprises 2000 Dalton (Da) polyethylene glycol. In further embodiments, the lipid-polyethylene glycol (lipid-PEG) conjugate is lipid-PEG-maleimide. In still further embodiments, the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof.

SNAs can range in size from about 1 nanometer (nm) to about 500 nm, about 1 nm to about 400 nm, about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In further aspects, the disclosure provides a plurality of SNAs, each SNA comprising one or more oligonucleotides attached thereto. Thus, in some embodiments, the size of the plurality of SNAs is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of SNAs) of the SNAs is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the SNAs, for example, the amount of surface area to which oligonucleotides may be attached as described herein. It will be understood that the foregoing diameters of SNAs can apply to the diameter of the nanoparticle core itself or to the diameter of the nanoparticle core and the one or more oligonucleotides attached thereto.

### Hydrophobic Anchor

In any of the aspects or embodiments of the disclosure, a spherical nucleic acid (SNA) is provided comprising: (a) a nanoparticle core; and (b) a shell of oligonucleotides attached to the nanoparticle core, wherein one or more oligonucleotides in the shell of oligonucleotides is attached to the nanoparticle core through a hydrophobic anchor comprising one or more dodecane (C12) subunits. Accordingly, one or more oligonucleotides in the shell of oligonucleotides comprises a hydrophobic anchor on its 5′ and/or 3′ end that facilitates intercalation of the one or more oligonucleotides in the nanoparticle core. In any of the aspects or embodiments of the disclosure, each oligonucleotide in the shell of oligonucleotides comprises a hydrophobic anchor on its 5′ and/or 3′ end that facilitates intercalation of each oligonucleotide in the shell of oligonucleotides in the nanoparticle core. In various embodiments, the hydrophobic anchor comprises 1 to about 20 dodecane subunits. Thus, in various embodiments, the hydrophobic anchor comprises or consists of 1, 2, 3, 4, 5, 6, 7, 8, 9, 10, 11, 12, 13, 14, 15, 16, 17, 18, 19, or 20 dodecane subunits. In further embodiments, the hydrophobic anchor comprises less than 1, 2, 3, 4, 5, 6, 7, 8, 9, 10, 11, 12, 13, 14, 15, 16, 17, 18, 19, or 20 dodecane subunits. In still further embodiments, the hydrophobic anchor comprises of at least 1, 2, 3, 4, 5, 6, 7, 8, 9, 10, 11, 12, 13, 14, 15, 16, 17, 18, 19, or 20 dodecane subunits. In any of the aspects or embodiments of the disclosure, the hydrophobic anchor comprises 9 dodecane subunits. In various embodiments, each oligonucleotide in the shell of oligonucleotides comprises a hydrophobic anchor. In various embodiments, about, less than about, or at least about 5%, 10%, 20%, 30%, 40%, 50%, 60%, 70% 80%, 90%, 95%, or 100% of oligonucleotides in the shell of oligonucleotides of a SNA comprises a hydrophobic anchor. In some embodiments, the hydrophobic anchor is attached to the 5′ end of an oligonucleotide in the shell of oligonucleotides. In some embodiments, the hydrophobic anchor is attached to the 3′ end of an oligonucleotide in the shell of oligonucleotides.

In any of the aspects or embodiments of the disclosure, the hydrophobic anchor comprises a structure as depicted below:

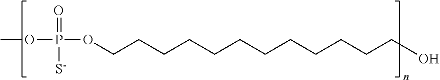

wherein n is about 1 to about 20.

Methods of attaching a hydrophobic anchor to an oligonucleotide. The following description is a non-limiting example of a method to attach a hydrophobic anchor to an oligonucleotide. Oligonucleotides (e.g., DNA) may be synthesized utilizing standard phosphoramidite chemistry with a phosphorothioate backbone. Cholesterol-terminated and thiol-terminated oligonucleotides may be synthesized on 3′-cholesteryl-TEG and 3′ thiol modifier C3 S-S supports, respectively. Following synthesis, DNA is cleaved and deprotected in an aqueous solution of 30 wt % ammonia and 40 wt % methylamine 1:1 (v/v) (Sigma-Aldrich) at 55° C. for 50 min. Oligonucleotides containing a C12 spacer (12-(4,4′-Dimethoxytrityloxy)dodecyl-1-[(2-cyanoethyl)-(N,N-diisopropyl)]-phosphoramidite) may be synthesized without a 5′ dimethoxytrityl (DMT) group and required lengthened (16 h) deprotection time at 55° C. to remove the Unylinker support. Cy5-labeled DNA may be deprotected under mild conditions (30 wt % ammonia for 20 h at room temperature). After deprotection, ammonia is evaporated under a stream of nitrogen gas and lyophilized. C12-containing strands may be purified by preparative-scale denaturing (8 M urea) polyacrylamide (15%, 19:1 acrylamide:bis, BioRad) gel electrophoresis (PAGE). 0.5 μmole of DNA in 0.5 mL water is mixed 1:1 with loading dye (8M urea with bromophenol blue) and loaded onto the gel which was run for 30 min at 175 V then ramped to 350 V for an additional 3 hours. Bands may be visualized with a 260 nm UV lamp against a thin-layer chromatography plate and the highest retention band isolated, crushed, washed 3× with water, lyophilized, and washed 3-4 times with water in a 3 kDa Amicron ultra-centrifugal filter (Millipore-Sigma) prior to identification of the desired product by matrix-assisted laser-desorption ionization time-of-flight (MALDI-ToF) spectroscopy (Bruker RapiFlex Tissue Typer). For MALDI-ToF, 0.5 μL sample is spotted with 0.5 μL of dihydroxyacetophenone matrix. Other oligonucleotides (e.g., DNAs) which contain a 5′ DMT are purified by reverse phase high-performance liquid chromatography with a gradient of 0.1M triethylammonium acetate (aq) to acetonitrile on a C18 or C4 (for Cy5 and cholesterolated DNAs) resin. Fractions with the greatest column retention are isolated, lyophilized, and the DMT group removed in 20% acetic acid (aq) for 1 h, washed 3× with ethyl acetate, and lyophilized again prior to resuspension in water and product identification by MALDI-ToF.

### Antigens

Spherical nucleic acids (SNAs) of the disclosure comprise, in various aspects and embodiments, one or more antigens. The antigen, in various embodiments, is a tumor antigen, a viral antigen, a bacterial antigen, or a combination thereof. In some embodiments, the viral antigen is a coronavirus antigen, an influenza virus, a herpes virus (e.g., herpes zoster), a human papilloma virus (HPV), a human immunodeficiency virus (HIV), measles, mumps, and Rubella (MMR), a variant of any of the foregoing, or a combination thereof. In further embodiments, the coronavirus is SARS-CoV-2 or a variant thereof. In related embodiments, the antigen is derived from a SARS-CoV-2 spike receptor binding domain or any variant thereof. In still further embodiments, the viral antigen is or is derived from SARS-CoV-2 envelope protein, SARS-CoV-2 nucleocapsid protein, SARS-CoV-2 membrane protein, a variant or fragment of any of the foregoing, or a combination thereof. As used herein, a “variant” refers to a genetic variant that comprises one or more mutations relative to a wild type amino acid sequence. Thus, in any of the aspects or embodiments of the disclosure, a viral antigen comprises or consists of a nucleotide or an amino acid sequence that is at least 80%, at least 85%, at least 90%, at least 95%, at least 99%, or 100% identical to a reference or wild type sequence.

In various embodiments, the tumor antigen is a melanoma related antigen, a HPV related antigen, a colon cancer antigen, a lymphoma antigen, a prostate cancer related antigen (e.g., prostate-specific membrane antigen), a glioblastoma antigen, an ovarian cancer related antigen, a breast cancer related antigen, a hepatocellular carcinoma related antigen, a lung cancer related antigen, a bowel cancer related antigen, or human papillomavirus (HPV) E7 nuclear protein.

In various embodiments, the antigen is encapsulated in the nanoparticle core, attached to one or more oligonucleotides in the shell of oligonucleotides through a linker, attached to the external surface of the nanoparticle core through a linker, or a combination thereof. In some embodiments, the antigen is attached through the linker to each oligonucleotide in the shell of oligonucleotides. In some embodiments, the antigen is attached through the linker to an oligonucleotide that is hybridized to an oligonucleotide in the shell of oligonucleotides that is attached to the nanoparticle core. Linkers and methods of associating antigens to SNAs are further described herein below.

### Oligonucleotides

The disclosure provides spherical nucleic acids (SNAs) comprising: (a) a nanoparticle core; and (b) a shell of oligonucleotides attached to the nanoparticle core, wherein one or more oligonucleotides in the shell of oligonucleotides is attached to the nanoparticle core through a hydrophobic anchor comprising one or more dodecane (C12) subunits. In various embodiments, the shell of oligonucleotides comprises an inhibitory oligonucleotide, an immunostimulatory oligonucleotide, a targeting oligonucleotide, or a combination thereof. Oligonucleotides contemplated for use according to the disclosure include those attached to a nanoparticle core through any means (e.g., covalent or non-covalent attachment). Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded. In any aspects or embodiments of the disclosure, an oligonucleotide comprises a detectable marker.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e., a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—CH₂—, —O—CH₂—CH=(including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N=(including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH=(including R⁵ when used as linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and ma re from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH2CH2CH2NH₂), 2′-allyl (2′-CH2—CH=CH2), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1 H-pyrimido[5 ,4-b][1,4]benzoxazin-2(3H)-one), phenothiazine cytidine (1 H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 5 nucleotides to about 1000 nucleotides in length. More specifically, an oligonucleotide of the disclosure is about 5 to about 1000 nucleotides in length, about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 1000 nucleotides in length, about 10 to about 900 nucleotides in length, about 10 to about 800 nucleotides in length, about 10 to about 700 nucleotides in length, about 10 to about 600 nucleotides in length, about 10 to about 500 nucleotides in length about 10 to about 450 nucleotides in length, about 10 to about 400 nucleotides in length, about 10 to about 350 nucleotides in length, about 10 to about 300 nucleotides in length, about 10 to about 250 nucleotides in length, about 10 to about 200 nucleotides in length, about 10 to about 150 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. In further embodiments, an oligonucleotide of the disclosure is about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In various embodiments, the shell of oligonucleotides attached to the exterior of the nanoparticle core of the SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality. For example, and without limitation, in some embodiments the shell of oligonucleotides comprises a plurality of immunostimulatory oligonucleotides, wherein one immunostimulatory oligonucleotide has a sequence that is different than at least one other immunostimulatory oligonucleotide in the plurality.

In some embodiments, one or more oligonucleotides in the shell of oligonucleotides comprises or consists of a (GGX)ₙ nucleotide sequence, wherein n is 2-20 and X is a nucleobase (A, C, T, G, or U). In some embodiments, the (GGX)ₙ nucleotide sequence is on the 5′ end of the one or more oligonucleotides. In some embodiments, the (GGX)ₙ nucleotide sequence is on the 3′ end of the one or more oligonucleotides. In some embodiments, one or more oligonucleotides in the shell of oligonucleotides comprises or consists of a (GGT)ₙ nucleotide sequence, wherein n is 2-20. In some embodiments, the (GGT)ₙ nucleotide sequence is on the 5′ end of the one or more oligonucleotides. In some embodiments, the (GGT)n nucleotide sequence is on the 3′ end of the one or more oligonucleotides.

In some embodiments, an oligonucleotide in the shell of oligonucleotides is a targeting oligonucleotide, such as an aptamer. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

Spacers. In some aspects and embodiments, one or more oligonucleotides in the shell of oligonucleotides that is attached to the nanoparticle core of a SNA comprise a spacer. “Spacer” as used herein means a moiety that serves to increase distance between the nanoparticle core and the oligonucleotide, or to increase distance between individual oligonucleotides when attached to the nanoparticle core in multiple copies, or to improve the synthesis of the SNA. Thus, spacers are contemplated being located between an oligonucleotide and the nanoparticle core.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the nanoparticle core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

SNA surface density. Generally, a surface density of oligonucleotides that is at least about 0.5 pmol/cm² will be adequate to provide a stable SNA. In further embodiments, a surface density of oligonucleotides that is at least about 1 pmol/cm², 1.5 pmol/cm², or 2 pmoles/cm² will be adequate to provide a stable SNA (e.g., LSNA or LNP-SNA). In some aspects, the surface density of a SNA of the disclosure is at least 15 pmoles/cm². Methods are also provided wherein the oligonucleotide is attached to the nanoparticle core of the SNA at a surface density of about 0.5 pmol/cm² to about 1000 pmol/cm², or about 2 pmol/cm² to about 200 pmol/cm², or about 10 pmol/cm² to about 100 pmol/cm². In some embodiments, the surface density is about 1.7 pmol/cm². In some embodiments, the surface density is about 2 pmol/cm². In further embodiments, the surface density is at least about 0.5 pmol/cm², at least about 0.6 pmol/cm², at least about 0.7 pmol/cm², at least about 0.8 pmol/cm², at least about 0.9 pmol/cm², at least about 1 pmol/mcg, at least about 1.5 pmol/cm², at least about 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. In further embodiments, the surface density is less than about 2 pmol/cm², less than about 3 pmol/cm², less than about 4 pmol/cm², less than about 5 pmol/cm², less than about 6 pmol/cm², less than about 7 pmol/cm², less than about 8 pmol/cm², less than about 9 pmol/cm², less than about 10 pmol/cm², less than about 15 pmol/cm², less than about 19 pmol/cm², less than about 20 pmol/cm², less than about 25 pmol/cm², less than about 30 pmol/cm², less than about 35 pmol/cm², less than about 40 pmol/cm², less than about 45 pmol/cm², less than about 50 pmol/cm², less than about 55 pmol/cm², less than about 60 pmol/cm², less than about 65 pmol/cm², less than about 70 pmol/cm², less than about 75 pmol/cm², less than about t 80 pmol/cm², less than about 85 pmol/cm², less than about 90 pmol/cm², less than about 95 pmol/cm², less than about 100 pmol/cm², less than about 125 pmol/cm², less than about 150 pmol/cm², less than about 175 pmol/cm², less than about 200 pmol/cm², less than about 250 pmol/cm², less than about 300 pmol/cm², less than about 350 pmol/cm², less than about 400 pmol/cm², less than about 450 pmol/cm², less than about 500 pmol/cm², less than about 550 pmol/cm², less than about 600 pmol/cm², less than about 650 pmol/cm², less than about 700 pmol/cm², less than about 750 pmol/cm², less than about 800 pmol/cm², less than about 850 pmol/cm², less than about 900 pmol/cm², less than about 950 pmol/cm², or less than about 1000 pmol/cm².

Alternatively, the density of oligonucleotide attached to the SNA is measured by the number of oligonucleotides attached to the SNA. With respect to the surface density of oligonucleotides attached to a SNA of the disclosure, it is contemplated that a SNA as described herein comprises or consists of about 1 to about 2,500, or about 1 to about 500 oligonucleotides on its surface. In various embodiments, a SNA comprises about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20, or about 75 to about 200, or about 75 to about 150, or about 100 to about 200, or about 150 to about 200 oligonucleotides in the shell of oligonucleotides attached to the nanoparticle core. In some embodiments, a SNA comprises about 80 to about 140 oligonucleotides in the shell of oligonucleotides attached to the nanoparticle core. In further embodiments, a SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the nanoparticle core. In further embodiments, a SNA consists of 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the nanoparticle core. In still further embodiments, the shell of oligonucleotides attached to the nanoparticle core of the SNA comprises 2, 3, 4, 5, 6, 7, 8, 9, 10, 11, 12, 13, 14, 15, 16, 17, 18, 19, 20, 50, 60, 70, 75, 80, 90, 100, 150, 160, 170, 175, 180, 190, 200, or more oligonucleotides. In some embodiments, the shell of oligonucleotides attached to the nanoparticle core of the SNA consists of 2, 3, 4, 5, 6, 7, 8, 9, 10, 11, 12, 13, 14, 15, 16, 17, 18, 19, 20, 30, 40, 50, 60, 70, 75, 80, 90, 100, 150, 160, 170, 175, 180, 190, or 200 oligonucleotides. In some embodiments, the shell of oligonucleotides comprises about 10 to about 80 oligonucleotides. In some embodiments, the shell of oligonucleotides comprises or consists of about 75 oligonucleotides. In some embodiments, the shell of oligonucleotides attached to the nanoparticle core of the SNA comprises or consists of 100 oligonucleotides. In some embodiments, the shell of oligonucleotides attached to the nanoparticle core of the SNA comprises or consists of 200 oligonucleotides. In some embodiments, the shell of oligonucleotides attached to the nanoparticle core of the SNA comprises or consists of 400 oligonucleotides.

Linkers. In various aspects and embodiments, the disclosure provides SNAs wherein one or more antigens are associated with and/or attached to the surface of a SNA via a linker. The linker can be, in various embodiments, a cleavable linker, a non-cleavable linker, a traceless linker, and a combination thereof.

The linker links the antigen to the oligonucleotide in the disclosed SNA or links the antigen to the surface of the SNA (i.e., Antigen-LINKER-Oligonucleotide or Antigen-LINKER). The oligonucleotide can be hybridized to another oligonucleotide attached to the SNA or can be directly attached to the SNA (e.g., via a hydrophobic anchor as described herein). Some specifically contemplated linkers include carbamate alkylene, carbamate alkylenearyl disulfide linkers, amide alkylene disulfide linkers, amide alkylenearyl disulfide linkers, thiol linkers, and amide alkylene succinimidyl linkers. In some cases, the linker comprises —NH—C(O)—O—C₂₋₅alkylene-S—S—CH₂₋₇alkylene- or —NH—C(O)—C₂₋₅alkylene-S—S—C₂₋₇alkylene-. The carbon alpha to the —S—S— moiety can be branched, e.g., —CHX—S—S— or —S—S—CHY— or a combination thereof, where X and Y are independently Me, Et, or iPr. The carbon alpha to the antigen can be branched, e.g., —CHX—C₂₋₄alkylene-S—S—, where X is Me, Et, or iPr. In some cases, the linker is —NH—C(O)—O—CH₂—ar—S—S—C₂₋₇alkylene-, and Ar is a meta- or para-substituted phenyl. In some cases, the linker is —NH—C(O)—C₂₋₄alkylene-N-succinimidyl-S—C₂₋₆alkylene-. Additional linkers include an SH linker, SM linker, SE linker, and SI linker.

### Uses of SNAs iN GENE REGULATION

In some aspects of the disclosure, the shell of oligonucleotides that is attached to the nanoparticle core comprises one or more inhibitory oligonucleotides designed to inhibit target gene expression. In some embodiments, each oligonucleotide in the shell of oligonucleotides attached to a SNA of the disclosure is an inhibitory oligonucleotide. In some embodiments, a SNA performs both a vaccine function and a gene inhibitory function. In such aspects, the shell of oligonucleotides that is attached to the nanoparticle core comprises one or more immunostimulatory oligonucleotides and one or more inhibitory oligonucleotides designed to inhibit target gene expression.

Methods for inhibiting gene product expression provided herein include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide.

In various aspects, the methods include use of an inhibitory oligonucleotide which is 100% complementary to the target polynucleotide, i.e., a perfect match, while in other aspects, the oligonucleotide is at least (meaning greater than or equal to) about 95% complementary to the polynucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the oligonucleotide to the extent that the oligonucleotide is able to achieve the desired degree of inhibition of a target gene product.

The percent complementarity is determined over the length of the oligonucleotide. For example, given an antisense compound in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

The oligonucleotide utilized in such methods is either RNA or DNA. The RNA can be an inhibitory oligonucleotide, such as an inhibitory RNA (RNAi) that performs a regulatory function, and in various embodiments is selected from the group consisting of a small inhibitory RNA (siRNA), a single-stranded RNA (ssRNA), and a ribozyme. Alternatively, the RNA is microRNA that performs a regulatory function. The DNA is, in some embodiments, an antisense-DNA. In some embodiments, the RNA is a piwi-interacting RNA (piRNA).

### Uses of SNAs in Immune Regulation

Toll-like receptors (TLRs) are a class of proteins, expressed in sentinel cells, that play a key role in regulation of innate immune system. The mammalian immune system uses two general strategies to combat diseases (e.g., infectious diseases). Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 8 and TLR 9 that respond to specific oligonucleotides are located inside special intracellular compartments, called endosomes. The mechanism of modulation of, for example and without limitation, TLR 8 and TLR 9 receptors, is based on DNA-protein interactions.

As described herein, synthetic immunostimulatory oligonucleotides that contain CpG motifs that are similar to those found in bacterial DNA stimulate a similar response of the TLR receptors. Thus, CpG oligonucleotides of the disclosure have the ability to function as TLR agonists. Other TLR agonists contemplated by the disclosure include, without limitation, single-stranded RNA and small molecules (e.g., R848 (Resiquimod)). Therefore, immunomodulatory (e.g., immunostimulatory) oligonucleotides have various potential therapeutic uses, including treatment of diseases (e.g., cancer).

Accordingly, in some embodiments, methods of utilizing SNAs as described herein for modulating toll-like receptors are disclosed. The method up-regulates the Toll-like-receptor activity through the use of a TLR agonist. The method comprises contacting a cell having a toll-like receptor with a SNA of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

### Methods of Inducing an Immune Response

The disclosure also includes methods for eliciting an immune response in a subject in need thereof, comprising administering to the subject an effective amount of a SNA (e.g., formulated as an antigenic composition) of the disclosure. In some embodiments, administering SNAs of the disclosure (SNAs comprising a hydrophobic anchor as described herein) to a subject results in an increased CD8 T cell response in the subject relative to the CD8 T cell response that is produced in a subject who was not administered the SNAs, or relative to the CD8 T cell response that is produced in a subject who was administered SNAs that did not comprise a hydrophobic anchor as described herein (e.g., a liposomal SNA comprising lipid anchors). In various embodiments, the CD8 T cell response in the subject is increased by about 2-fold, 3-fold, 4-fold, 5-fold, or more in the subject that was administered a SNA of the disclosure relative to the CD8 T cell response that is produced in a subject who was not administered the SNAs, or relative to the CD8 T cell response that is produced in a subject who was administered SNAs that did not comprise a hydrophobic anchor as described herein In various embodiments, administering SNAs of the disclosure (e.g., formulated as a composition, pharmaceutical formulation, or antigenic composition) to a subject results in an increase in the amount of neutralizing antibodies against the antigen(s) that is produced in the subject relative to the amount of neutralizing antibodies against the antigen(s) that is produced in a subject who was not administered the SNAs, or relative to the amount of neutralizing antibodies against the antigen(s) that is produced in a subject who was administered SNAs that did not comprise a hydrophobic anchor as described herein. In further embodiments, the increase is a 2-fold increase, a 5-fold increase, a 10-fold increase, a 50-fold increase, a 100-fold increase, a 200-fold increase, a 500-fold increase, a 700-fold increase, or a 1000-fold increase. In various embodiments, and as demonstrated herein (see, e.g., Example 3) administering SNAs of the disclosure (e.g., formulated as a composition, pharmaceutical formulation, or antigenic composition) to a subject does not result in a significant increase in cytokine production (e.g., TNF-α, IFN-γ, IL-6, IL-12p70, IL-1β, IL-4, or IL-5) compared to administration of an immunostimulatory oligonucleotide alone. In various embodiments, administration of an immunostimulatory oligonucleotide alone results in about or at least about a 2-fold, 5-fold, 10-fold, 20-fold, 50-fold, 75-fold, or 100-fold increase in cytokine level compared to administration of a SNA of the disclosure. In further embodiments, administration of a SNA of the disclosure comprising a hydrophobic anchor results in a less than or equal to about 25%, 20%, 15%, 10%, 5%, 3%, 2%, or 1% increase in serum level of a cytokine (e.g., interleukin-2 (IL-2), interleukin-6 (IL-6), interleukin-10 (IL-10), C-X-C Motif Chemokine Ligand 1 (CXCL1), tumor necrosis factor α (TNF-α), interleukin-1 (IL-1), interferon, or a combination thereof) in the subject. In still further embodiments, administration of a SNA of the disclosure comprising a hydrophobic anchor results in a less than or equal to about 25%, 20%, 15%, 10%, 5%, 3%, 2%, or 1% increase in serum level of a cytokine (e.g., interleukin-2 (IL-2), interleukin-6 (IL-6), interleukin-10 (IL-10), C-X-C Motif Chemokine Ligand 1 (CXCL1), tumor necrosis factor α (TNF-α), interleukin-1 (IL-1), interferon, or a combination thereof) in the subject compared to serum level of a cytokine in the subject in the absence of administration of a SNA of the disclosure, or compared to serum level of a cytokine in the subject following administration of a SNA that did not comprise a hydrophobic anchor as described herein. In further embodiments, administering SNAs of the disclosure (e.g., formulated as a composition, pharmaceutical formulation, or antigenic composition) to a subject results in dendritic cell activation. Dendritic cell activation may be measured by, e.g., increased expression of CD86, CD83, and/or CD80 relative to expression in the absence of the SNA. Following administration of a SNA of the disclosure, the expression of CD86, CD83, and/or CD80 may be increased about 2-fold, 5-fold, 10-fold, or 20-fold compared to expression of CD86, CD83, and/or CD80 in the absence of administration of a SNA of the disclosure, or compared to expression of CD86, CD83, and/or CD80 following administration of a SNA that did not comprise a hydrophobic anchor as described herein.

In further embodiments, SNAs of the disclosure activate human peripheral blood mononuclear cells and generate an antibody response against one or more antigens as described herein. In some embodiments, the antibody response is a total antigen-specific antibody response. In further embodiments, administering SNAs of the disclosure (e.g., formulated as a composition, pharmaceutical formulation, or antigenic composition) to a subject results in an increase in the amount of total antigen-specific antibodies against the antigen(s) that is produced in the subject relative to the amount of total antigen-specific antibodies against the antigen(s) that is produced in a subject who was not administered the SNAs. In further embodiments, the increase is a 2-fold increase, a 5-fold increase, a 10-fold increase, a 50-fold increase, a 100-fold increase, a 200-fold increase, a 500-fold increase, a 700-fold increase, or a 1000-fold increase. A “total antigen-specific antibody response” is a measure of all of the antibodies (including neutralizing and non-neutralizing antibodies) that bind to a particular antigen.

The immune response raised by the methods of the present disclosure generally includes an antibody response, preferably a neutralizing antibody response, maturation and memory of T and B cells, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the SNA as disclosed herein generates an immune response and preferably treats a disease (e.g., cancer, infectious disease) as described herein. Methods for assessing antibody responses after administration of an antigenic composition (immunization or vaccination) are known in the art and/or described herein. In some embodiments, the immune response comprises a T cell-mediated response (e.g., peptide-specific response such as a proliferative response or a cytokine response). In any of the aspects or embodiments of the disclosure, the immune response comprises both a B cell and a T cell response. Antigenic compositions can be administered in a number of suitable ways, such as intramuscular injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, or a subcutaneous prime and a subcutaneous boost. Administration of more than one dose (typically two doses) is particularly useful in immunologically naive subjects or subjects of a hyporesponsive population (e.g., diabetics, or subjects with chronic kidney disease (e.g., dialysis patients)). In various embodiments, the second dose is administered about or at least about 2 weeks after the first dose. Multiple doses will typically be administered at least 1 week apart (e.g., about 2 weeks, about 3 weeks, about 4 weeks, about 6 weeks, about 8 weeks, about 10 weeks, about 12 weeks, or about 16 weeks). In some embodiments, multiple doses are administered from one, two, three, four or five months apart. Antigenic compositions of the present disclosure may be administered to patients at substantially the same time as (e.g., during the same medical consultation or visit to a healthcare professional) other vaccines.

### Uses of SNAs to Treat a Disorder

In some aspects, a SNA of the disclosure is used to treat a disorder. Thus, in some aspects, the disclosure provides methods of treating a disorder comprising administering an effective amount of a SNA of the disclosure to a subject (e.g., a human subject) in need thereof, wherein the administering treats the disorder. In some aspects, the disclosure provides methods of treating a cancer comprising administering to a subject (e.g., a human subject) an effective amount of a SNA of the disclosure, thereby treating the cancer in the subject. In various embodiments, the cancer is bladder cancer, breast cancer, cervical cancer, colon cancer, rectal cancer, endometrial cancer, glioblastoma, kidney cancer, leukemia, liver cancer, lung cancer, melanoma, lymphoma, non-Hodgkin lymphoma, osteocarcinoma, ovarian cancer, pancreatic cancer, prostate cancer, thyroid cancer, and human papilloma virus-induced cancer, or a combination thereof.

In some aspects, the disclosure provides methods of treating an infectious disease comprising administering to a subject (e.g., a human subject) an effective amount of a SNA of the disclosure, thereby treating the infectious disease in the subject. In various embodiments, the infectious disease is a viral infection, a bacterial infection, or a combination thereof.

### Therapeutic Agents

In any of the aspects or embodiments of the disclosure, a therapeutic agent is administered separately from a SNA of the disclosure. Thus, in various embodiments, a therapeutic agent is administered before, after, or concurrently with a SNA of the disclosure to treat a disorder (e.g., cancer).

A therapeutic agent may also be associated with a SNA of the disclosure. Thus, in some aspects, the SNAs provided herein optionally further comprise a therapeutic agent, or a plurality thereof. The therapeutic agent is, in various embodiments, simply associated with an oligonucleotide in the shell of oligonucleotides attached to the exterior of the nanoparticle core of the SNA, and/or the therapeutic agent is associated with the nanoparticle core of the SNA, and/or the therapeutic agent is encapsulated in the nanoparticle core of the SNA. In some embodiments, the therapeutic agent is associated with the end of an oligonucleotide in the shell of oligonucleotides that is not attached to the nanoparticle core (e.g., if the oligonucleotide is attached to the nanoparticle core through its 3′ end, then the therapeutic agent is associated with the 5′ end of the oligonucleotide). Alternatively, in some embodiments, the therapeutic agent is associated with the end of an oligonucleotide in the shell of oligonucleotides that is attached to the nanoparticle core (e.g., if the oligonucleotide is attached to the nanoparticle core through its 3′ end, then the therapeutic agent is associated with the 3′ end of the oligonucleotide). In some embodiments, the therapeutic agent is covalently associated with an oligonucleotide in the shell of oligonucleotides that is attached to the exterior of the nanoparticle core of the SNA. In some embodiments, the therapeutic agent is non-covalently associated with an oligonucleotide in the shell of oligonucleotides that is attached to the exterior of the nanoparticle core of the SNA. However, it is understood that the disclosure provides SNAs wherein one or more therapeutic agents are both covalently and non-covalently associated with oligonucleotides in the shell of oligonucleotides that is attached to the exterior of the lipid nanoparticle core of the SNA. It will also be understood that non-covalent associations include hybridization, protein binding, and/or hydrophobic interactions.

Therapeutic agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof. In various embodiments, the therapeutic agent is an anti-programmed cell death protein 1 (PD-1) antibody, an anti-programmed death-ligand 1 (PD-L1) antibody, a cytotoxic T lymphocyte antigen 4 (CTLA-4) antibody, or a combination thereof.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

The following examples are given merely to illustrate the present disclosure and not in any way to limit its scope.

### EXAMPLES

### Example 1

This example demonstrates that compared to a conventional cholesterol anchor, adding up to 9-12 carbon (C12) spacers improved the serum half-life of LSNAs 50-fold and increased the loading of oligonucleotides on a liposome to 1.75 pmol/cm\hat{ }2. When comprised of an immunostimulatory CpG motif, these SNAs more potently activated dendritic cells in vitro and promoted enhanced cytotoxic T Cell immunity towards multiple antigenic targets in vivo.

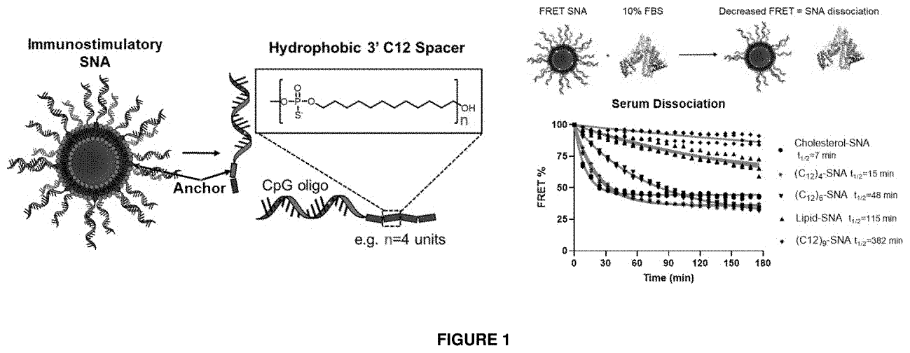

DNA with tunable hydrophobicity was synthesized on a solid support using phosphoramidite chemistry with a Spacer C12 CE phosphoramidite purchased from Glen Research. 1-10 of these units were added to the 3′ end of an immunostimulatory CpG motif oligonucleotide, cleaved from the solid support, and purified by denaturing polyacrylamide gel electrophoresis (PAGE). Successful synthesis of the product was verified by matrix-assisted laser desorption ionization spectroscopy time-of-flight (MALDI-ToF) mass spectrometry. Liposomes comprised of 1,2-dioleoyl-sn-glycero-3-phosphocholine lipid were synthesized by freeze thawing 10-50 mg of dried lipid in phosphate buffered saline (PBS) with or without dissolved peptide (1 mg/mL) in cases where the construct was designed to elicit an adaptive immune response towards the peptide antigen. Crude liposomes were then passed through a series of extrusion filters to generate 50 nm liposomes whose hydrodynamic size was verified using dynamic light scattering (DLS). Unencapsulated peptide was removed by dialysis, and the liposome concentration was determined using a commercially available phosphatidylcholine assay. Immunostimulatory LSNAs (FIG. 1) were synthesized by mixing oligonucleotides with tunable hydrophobicity with preformed liposomes, briefly sonicating the solution and then shaking the solution for 4 hours at 37° C. and stored at 4° C.

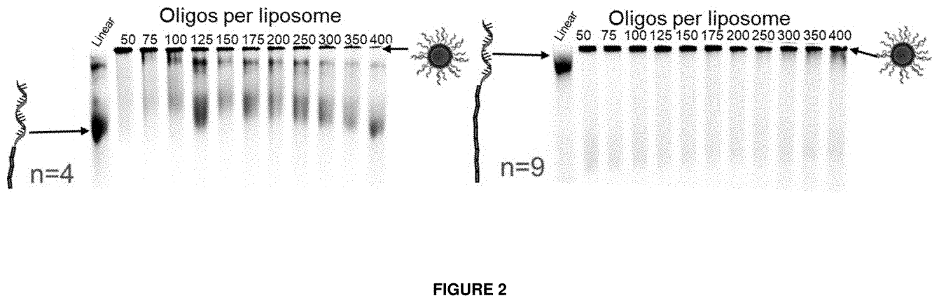

To test serum stability (FIG. 1), LSNAs were synthesized as described above with interior cyanine 5 labels on the oligonucleotide and 1% 1,2-dioleoyl-sn-glycero-3-phosphoethanolamine-N-(lissamine rhodamine B sulfonyl) (ammonium salt) lipid in the core of the liposome. FRET was measured by exciting the rhodamine dye and measuring Cy5 fluorescence over time during incubation of the LSNA with 10% heat inactivated fetal bovine serum. To determine the loading of oligonucleotides (FIG. 2) LSNAs were synthesized with decreasing amounts of liposome, holding the amount of DNA constant to change the ratio of oligonucleotide:liposome. These products were then run on a 2% agarose gel and the ratio at which a free oligonucleotide band was observed was determined to be the maximum loading for that oligonucleotide.

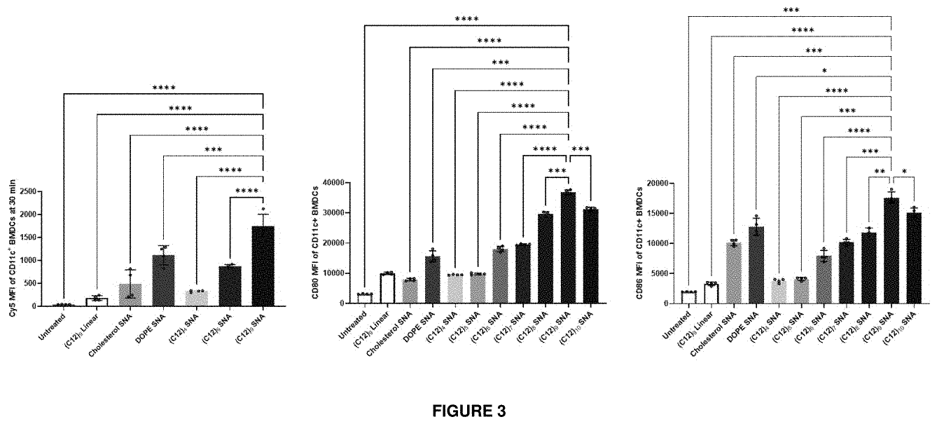

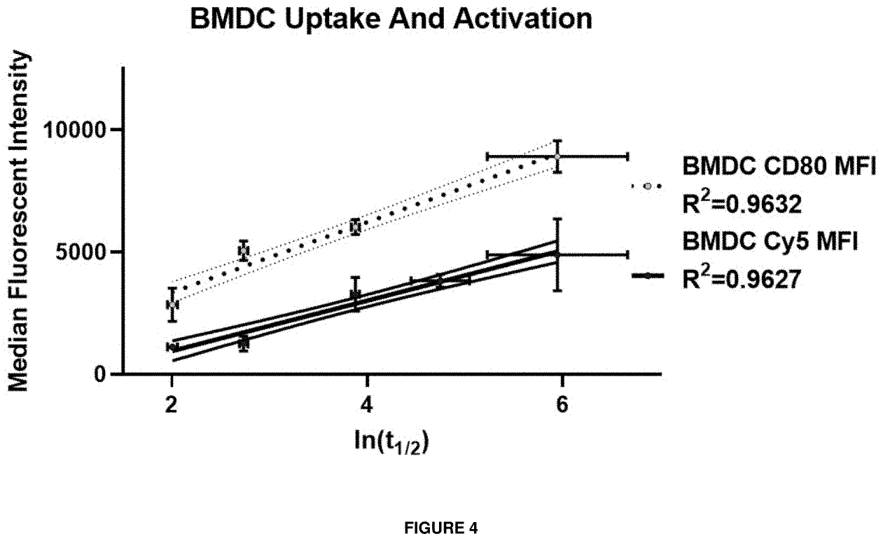

LSNA uptake and activation of bone-marrow derived dendritic cells (BMDCs) (FIG. 3) was determined by incubating 2.5×10\hat{ }5 cells with LSNAs at 0.1 or 2 uM respectively. BMDCs were generated by obtaining murine stem cells from the femur and tibia and incubating these cells with 40 ng/mL granulocyte-macrophage colony-stimulating factor (GMCSF) for 6 days. On day 6, cells were treated with LSNA (30 min. for uptake, 24 hr for activation) and stained with fluorophore-conjugated antibodies specific for CD11c, CD80, and CD86. Expression of these markers and uptake was measured by flow cytometry. Development of this tunable anchor also allowed for the determination that the relationship between LSNA stability and DC uptake and activation is linear when the LSNA half-life in serum is between 7 and 382 minutes (FIG. 4).

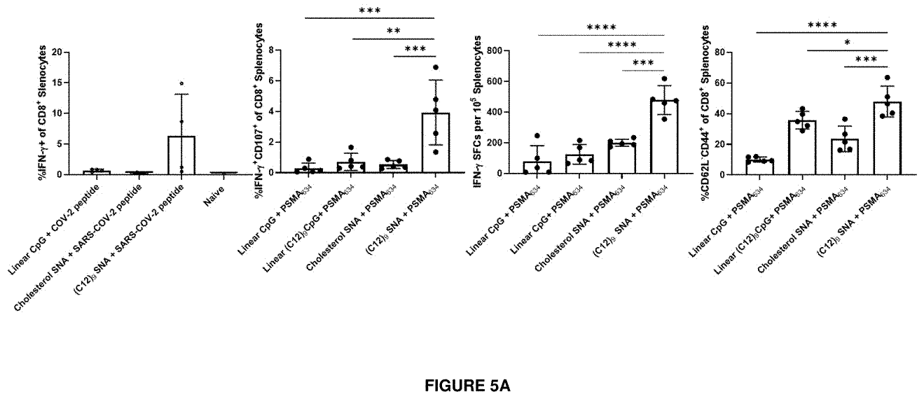

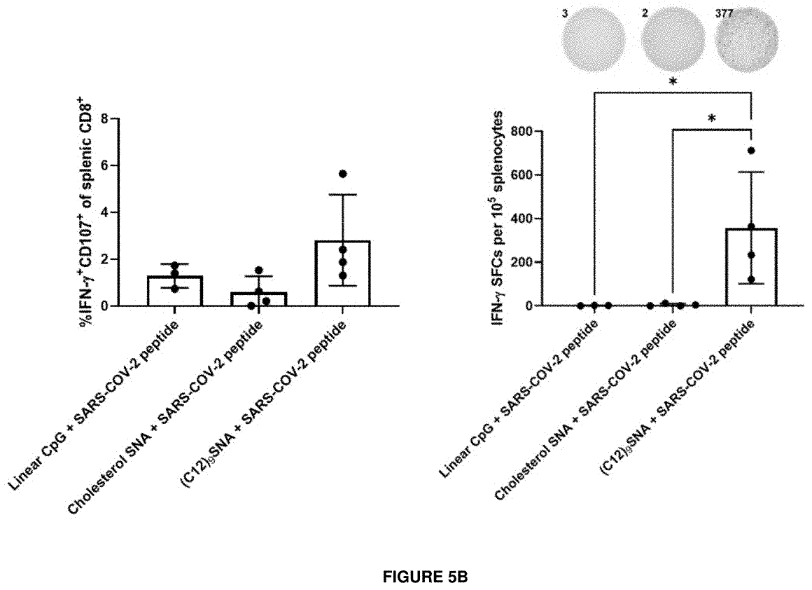

Mice were vaccinated fortnightly three times with LSNAs (6 nmol CpG oligo and peptide) or a mixture of linear CpG DNA with the 9 C12 anchor and the peptide to assess antigen-specific immunity built by mice vaccinated with the new LSNA design (FIG. 5A, 5B). The SARS-COV-2 peptide was identified as a conserved region of the SARS-COV spike protein (N-VLNDILSRL-C(SEQ ID NO: 1)) with affinity for human MHCl. AAD transgenic mice containing a gene insert for human MHCI were used to assess the SARS-COV-2 peptide vaccinated mice while wild type C57BL/6 mice were used to assess the immune response to prostate specific-membrane antigen (PSMA634, N-SAVKNFTEI-C (SEQ ID NO: 2)). On day 35, spleens were collected and a single cell suspension of splenocytes was incubated with the peptide vaccinated against to assess antigen-specific interferon gamma secretion by CD8 T Cells, measured both by flow cytometry and Enzyme-linked ImmunoSpot Assay (ELISA). In PSMA634 vaccinated mice, the CD8 T Cell memory population in the spleen was also quantified via flow cytometry using the CD62L-CD44+ positive population as an identifier of memory T cells.

### Example 2

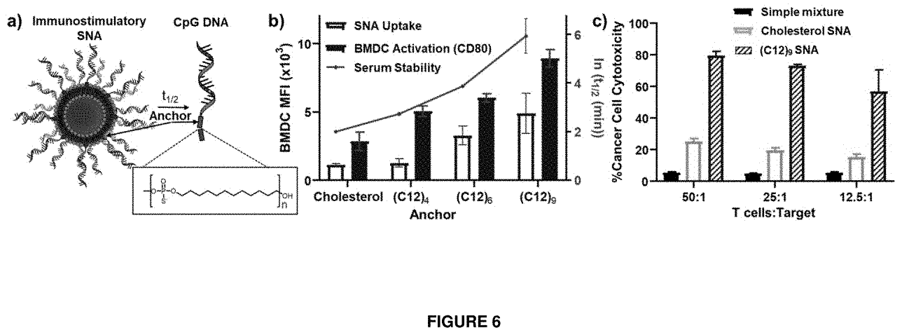

Materials and Methods: SNAs with liposomal cores were assembled through hydrophobic interactions between the liposome and an anchor on the 3′ terminus of the DNA (FIG. 6a). The anchor is a series of dodecane (C12) subunits (FIG. 6a) or cholesterol incorporated during standard DNA synthesis. The dissociation half-life (t_{1/2},) of DNA from the liposome was assessed via Förster Resonance Energy Transfer (FRET) assays (FIG. 6b). SNA immunostimulation was assessed in vitro by quantifying murine bone marrow dendritic cell (BMDC) uptake and activation, and in vivo CD8+ T cell expansion, cytokine secretion, and T cell killing by flow cytometry (FIG. 6b, c).

Results and Discussion: In serum, the DNA dissociation half-life from the liposome surface increased with increasing hydrophobicity of the anchor until no apparent decay was observed with the (C12)₉ anchor (exponential fit R²=0.34, FIG. 6b. The stability of the SNA enhanced the immune response as evidenced by a linear correlation between the log-transform of serum half-life (In(t_{1/2})), cellular uptake (R²=0.96), and CD80 expression (R²'0.96). SNA activation of APCs can therefore be increased by enhancing stability of these structures. In vivo, the most stable (C12)₉ anchored SNAs enhanced clonal CD8⁺ T cell expansion IFN-γ expression and cancer cell killing (FIG. 6c) compared to conventional cholesterol-anchored SNAs. T cells raised from (C12)₉ vaccination exhibited great cytolytic potential, even at high dilution (12.5 T cells:target), and were nearly 4 times more potent than cholesterol-anchored SNAs (p=0.0016, Two-way ANOVA) and 12 times more potent than a simple mixture of CpG DNA and OVA1 peptide (p=0.0005). (C12)₉ SNAs were also used to generate immune responses towards prostate-specific membrane antigen to target prostate cancer.

Conclusions: Increasing the hydrophobicity of the anchor by multiple additions of the C12 unit led to profound enhancements in SNA stability and augmentation of their immunogenic properties in vitro and in vivo. This work revealed new relationships between SNA stability and its immunogenic properties, which will aid in rational vaccine design. Due to the tunability of these interactions, this approach can be utilized in systems where control over ligand release is desired, such as sheddable stealth liposomes with tunable biodistribution, or control over the liposome's biophysical properties.

### Example 3

To gain greater insight into the biological mechanisms responsible for the reductions in tumor growth for SNA-treated mice, the proinflammatory serum cytokine profile was characterized after administration of either linear CpG or SNA adjuvants. Prior work has shown that linear CpG administration can lead to rapid cytokine release which upon repeated administration can attenuate the generation of an effective immune response [Agren J, Thiemermann C, Foster S J, Wang J E, Aasen A O. Cytokine responses to CpG DNA in human leukocytes. Scand J Immunol. 2006 July; 64(1):61-8. doi: 10.1111/j.1365-3083.2006.01779.x. PMID: 16784492]. Additionally, the excessive activation of the immune system can lead to severe, possibly fatal systemic inflammation [Heikenwalder M, Polymenidou M, Junt T, et al. Lymphoid follicle destruction and immunosuppression after repeated CpG oligodeoxynucleotide administration. Nat Med. 2004; 10(2)187-192. doi:10.1038/nm987; Sparwasser T, Hültner L, Koch E S, Luz A, Lipford G B, Wagner H. Immunostimulatory CpG-oligodeoxynucleotides cause extramedullary murine hemopoiesis. J Immunol. 1999; 162(4):2368-2374; Sparwasser T, Miethke T, Lipford G, et al. Bacterial DNA causes septic shock. Nature. 1997;386(6623):336-337. doi:10.1038/386336a0; Sparwasser T, Miethke T, Lipford G, et al. Macrophages sense pathogens via DNA motifs: induction of tumor necrosis factor-alpha-mediated shock. Eur J Immunol. 1997; 27(7):1671-1679. doi:10.1002/eji.1830270712]. It was hypothesized that the SNA architecture would provide a more targeted delivery of CpG with reduced risk of acute cytokine release since nanostructures more efficiently target lymph nodes [Reddy S T, van der Vlies A J, Simeoni E, et al. Exploiting lymphatic transport and complement activation in nanoparticle vaccines. Nat Biotechnol. 2007; 25(10):1159-1164. doi:10.1038/nbt1332]. C57BL6/J mice were treated with a standard CpG dose (6 nmol) of either linear CpG, cholesterol SNAs, or (C12)₉ SNAs. Cytokines from the serum of these mice were then measured with a Meso-Scale Diagnostics (MSD) multiassay system.

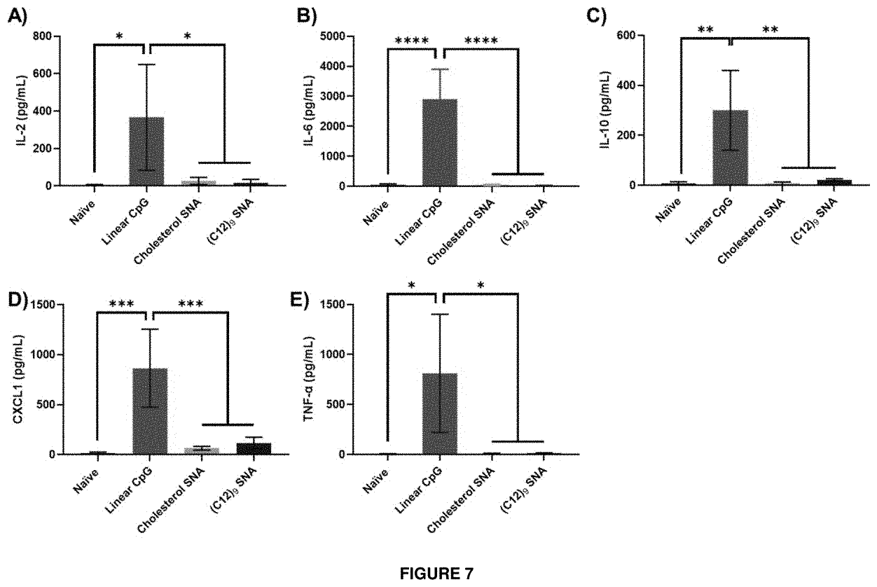

Analysis of systemic cytokine secretion shortly after treatment (1 h) revealed a spike in several proinflammatory cytokines in mice treated with linear CpG, but not in naïve mice or mice treated with SNAs (FIG. 7). Compared to naïve mice, linear CpG induced a 66-fold increase in IL-2 concentration (FIG. 7A), a 73-fold increase in IL-6 concentration (FIG. 7B), a 60-fold increase in IL-10 concentration (FIG. 7C), a 185-fold increase in TNF-α concentration (FIG. 7D), and a 60-fold increase in concentration of the chemokine CXCL1 (FIG. 7E). Mice treated with either cholesterol or (C12)₉ SNAs did not show a significant increase in serum cytokine levels compared to naïve mice. Significant increases were not observed in any treatment group for the cytokines IFN-γ, IL-12p70, IL-1β, IL-4, or IL-5 and cytokine levels measured 4 h after treatment were reduced to near-baseline levels. These data demonstrated the greater safety of SNA therapies due to their ability to deliver effective payloads with reduced systemic release of proinflammatory cytokines, which can lead to the generation of potentially fatal cytokine storms. The reduced secretion of IL-6 and TNF-α is especially significant due to the central role these cytokines play in the generation of cytokine storms.

### Example 4

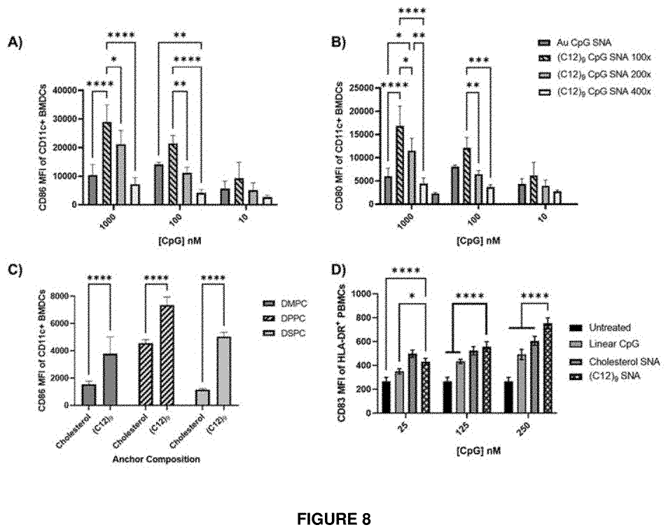

To gain further insight into the potency of (C12)₉ SNA activation of dendritic cells, the BMDC activation of (C12)₉ SNAs with both Au-core SNAs were compared with liposomal SNAs comprised of lipids with increasing melting transition temperatures. In prior work, Au SNAs were shown to more potently activate TLR9 due to the superior biostability of the Au-thiol bond. Since the maximum achievable oligonucleotide density is different between these two constructs, the oligonucleotide density of the (C12)₉ SNA was also varied to understand the effect of DNA shell density on the SNA's immunostimulatory effects. (C12)₉ SNAs with 100 ((C12)₉ SNA 100×), 200 ((C12)₉ SNA 200×), and 400 ((C12)₉ SNA 400×) DNA per 50 nm liposome were synthesized as well as Au SNAs which contained 250 CpG DNA per 13 nm Au particle (Au SNA 250×). Intriguingly, (C12)₉ SNA 100×, with the lowest tested DNA density, resulted in the greatest activation of both CD86 (FIG. 8A) and CD80 (FIG. 8B) at the two highest tested concentrations (1000 and 100 nM). The greatest degree of BMDC activation was observed at 1000 nM, at this concentration (C12)₉ SNA 100× treatment resulted in 2.7-fold, 1.4-fold, and 4-fold greater expression of CD86 than Au SNA 250×, (C12)₉ SNA 200×, and (C12)₉ SNA 400×, respectively. These trends are also apparent for CD80 expression suggesting that oligonucleotide density is a critical parameter for TLR9 activation and that a balance must be struck between having a high enough density for SNA uptake while being able to activate TLR9 to an optimal extent. Importantly, this result also demonstrated that stably designed liposomal SNAs can lead to greater BMDC activation than Au SNAs which are highly resistant to biological degradation. Since the core material of liposomal SNAs has also been shown to impact the stability of the DNA shell, this parameter was also varied by synthesizing liposomes comprised of 1,2-dimyristoyl-sn-glycero-3-phosphocholine (DMPC), 1,2-dipalmitoyl-sn-glycero-3-phosphocholine (DPPC), and 1,2-distearoyl-sn-glycero-3-phosphocholine (DSPC) with cholesterol or (C12)₉ anchored CpG and compared the activation profile of BMDCs with these two anchors at 1 μM concentration of CpG (FIG. 8C). In agreement with prior studies it was observed that the DPPC core led to the greatest enhancement of the SNAs immunostimulation. Additionally, for each type of liposome core the (C12)₉ anchor chemistry resulted in greater activation than the commonly employed cholesterol anchor. (C12)₉ anchored SNAs resulted in 2.5-fold, 1.6-fold, and 4.4-fold enhancements in CD86 expression compared to cholesterol-anchored SNAs comprised of DMPC, DPPC, and DSPC cores, respectively. This result demonstrated the generality of the (C12)₉ anchor chemistry for use with multiple types of liposomal formulations and its ability to consistently upregulate the SNAs biological properties. To demonstrate the potential for translation of the (C12)₉ anchor chemistry in clinical applications , SNAs with a sequence that activates human TLR9 (CpG 7909) were synthesized and incubated SNAs or linear CpG with human peripheral blood monocytes (PBMCs) pooled from multiple donors (FIG. 8D). The activation of HLA-DR+ antigen-presenting cells was measured by the expression of CD83, a marker that identifies matured human dendritic cells in blood. A dose-dependent increase in CD83 expression was observed with the (C12)₉ SNAs exhibiting the highest immunostimulatory potential at the highest tested concentration. At low concentrations of CpG 7909 (25 and 125 nM), (C12)₉ SNAs upregulate the expression of CD83 to a greater extent than linear CpG and untreated PBMCs. At higher concentrations (250 nM), (C12)₉ SNAs outperformed both of these adjuvants which have been utilized in human clinical trials (NCT00043407, NCT00070629, and NCT00233506) with a 2.8 fold enhancement in CD83 expression above untreated PBMCs, a 1.5 fold enhancement compared to linear CpG 7909, and a 1.2 fold increase compared to cholesterol SNAs.

### Example 5

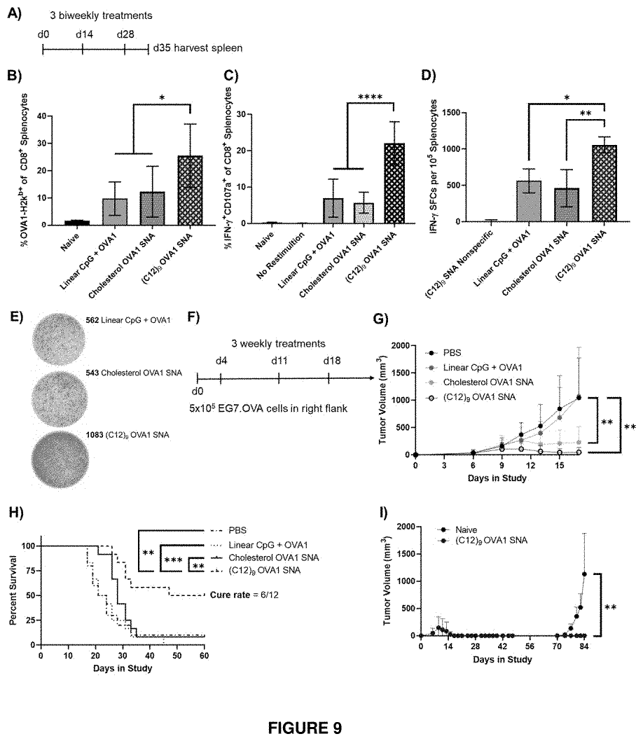

It was next evaluated how enhancements in in vitro SNA potency impacted in vivo raised immune responses. To evaluate the adaptive immune response initiated by SNA vaccination, the ubiquitous OVA1 model antigen (OVA₂₅₇₋₂₆₃ from the ovalbumin protein) was included either as a simple mixture of adjuvant and antigen (Linear CpG+OVA1) or included in the liposomal SNA structure with different anchor chemistries (cholesterol OVA1 SNA or (C12)₉ OVA1 SNAs. Mice were vaccinated with SNAs containing the OVA1 peptide and the (C12)₉ anchor ((C12)₉ OVA1 SNA), the cholesterol anchor (Cholesterol OVA1 SNA) since cholesterol SNAs have already been established as a powerful platform for cancer vaccination (clinical trial identifiers NCT03684785 and NCT03086278), or a mixture of linear CpG 1826 and OVA1. A biweekly vaccination schedule (FIG. 9A) was followed where C57BL/6J mice were injected subcutaneously (SQ) in the abdomen to promote drainage to the inguinal lymph nodes. On day 35, the mice were sacrificed, and splenocytes were isolated to analyze the impact of treatment on raising an immune response.

Consistent with the in vitro results where the (C12)₉ SNAs promoted the strongest DC activation, (C12)₉ OVA1 SNA vaccination also led to the most robust T cell immune responses in vivo. The CD8⁺ splenic T cells raised from (C12)₉ OVA1 SNA vaccination had a greater frequency of cells with T-cell receptors (TCRs) that recognize the OVA1 antigen (FIG. 9B). Over 25% of CD8⁺ TCRs were positive for the OVA1 peptide, a 2-fold enrichment in OVA1-specific T cells above cholesterol OVA1 SNA treated mice, a 2.6 fold enrichment above the mixture of linear CpG and OVA1, and a 16-fold increase compared to naïve mice. To assess the phenotype T cells raised from vaccination, the frequency of polyfunctional T cells that produced the effector cytokine IFN-γ and surface marker CD107a, which identifies degranulation [Aktas E, Kucuksezer U C, Bilgic S, Erten G, Deniz G. Relationship between CD107a expression and cytotoxic activity. Cell Immunol. 2009;254(2):149-154. doi:10.1016/j.cellimm.2008.08.007], in response to restimulation of splenocytes with OVA1 peptide was also quantified (FIG. 9C). (C12)₉ OVA1 SNA vaccination led to the strongest immune response, with 22% of the CD8⁺ T cells producing both IFN-γ and CD107a, a 3.9-fold increase compared to cholesterol SNAs, and a 3.2-fold increase above the mixture of linear CpG and OVA1. (C12)₉-OVA1 SNA T cells that were not restimulated with OVA1 peptide did not produce polyfunctional T cells, highlighting the specificity of the (C12)₉ SNA vaccination. The antigen-specific secretion of IFN-γ was assessed through an Enzyme-Linked Immunosorbent Spot (ELISpot) assay (FIG. 9D,E). (C12)₉ OVA1 SNA vaccination resulted in 2.3-fold more spot-forming cells (SFCs) than cholesterol OVA1 SNAs and a 1.9-fold higher number of SFCs than the CpG and OVA1 mixture. Together, these results demonstrated the superior ability of (C12)₉ SNA mediated immunotherapy to orchestrate an antigen-specific effector CD8⁺ T cell response in vivo.

Motivated by the robust immune responses generated by (C12)₉ OVA1 SNA vaccination, the efficacy of these SNAs was determined against an established E.G7-OVA lymphoma, which expresses the entire ovalbumin protein [Moore M W, Carbone F R, Bevan M J. Introduction of soluble protein into the class I pathway of antigen processing and presentation. Cell. 1988; 54(6):777-785. doi:10.1016/s0092-8674(88)91043-4]. Mice were inoculated with 5× 10⁵ lymphoma cells in the right hind flank and treated weekly with equimolar doses of either (C12)₉ OVA1 SNA, cholesterol OVA1 SNA, or a mixture of CpG and OVA1 (FIG. 9F). Both (C12)₉ OVA1 SNAs and cholesterol OVA1 SNAs were able to control tumor growth at early stages, resulting in 21.9-fold and a 4.6 fold reductions in tumor volumes compared to saline-treated mice on day 17, respectively (FIG. 9G). However, (C12)₉ OVA1 SNA treatment proved superior to the cholesterol OVA1 SNA treatment in the long term, likely due to the enhanced antitumor activity of the polyfunctional T cells raised from (C12)₉ OVA1 SNA vaccination. (C12)₉ OVA1 SNA treatment resulted in a significant extension of median survival (53.5 d) compared to cholesterol OVA1 SNA (28.5 d), linear CpG and OVA1 (22.5 d), and the saline control (22.5 days) (FIG. 9H). A promising indicator of the potent T cell memory imparted by (C12)₉ OVA1 SNAs, 6 of the 12 mice treated with (C12)₉ OVA1 SNAs did not exhibit tumor growth at the end of the study (d 70). The 6 surviving mice were then challenged with an additional injection of 5×10⁵ E.G7-OVA cells; none of these mice grew additional tumors (FIG. 9I).

### REFERENCES

1. Cassandra E. Callmann, Caroline D. Kusmierz, Jasper W. Dittmar, Leah Broger, and Chad A. Mirkin. (2021) Impact of Liposomal Spherical Nucleic Acid Structure on Immunotherapeutic Function ACS Central Science Article ASAP. DOI: 10.1021/acscentsci.1c00181
2. Qin, L.; Wang, S.; Dominguez, D.; Long, A.; Chen, S.; Fan, J.; Ahn, J.; Skakuj, K.; Huang, Z.; Lee, A.; Mirkin, C.; Zhang, B. (2020) Development of Spherical Nucleic Acids for Prostate Cancer Immunotherapy. Frontiers in Immunology, 11, 1333.
3. Wang, S.; Qin, L.; Yamankurt, G.; Skakuj, K.; Huang, Z.; Chen, P.; Dominguez, D.; Lee, A.; Zhang, B.; Mirkin, C. A. (2019). Rational vaccinology with spherical nucleic acids. Proceedings of the National Academy of Sciences, 116(21), 10473-10481.
4. Meckes, B.; Banga, R. J.; Nguyen, S. T.; Mirkin, C. A.; (2018) Enhancing the Stability and Immunomodulatory Activity of Liposomal Spherical Nucleic Acids through Lipid-Tail DNA Modifications. 14(5), 1702909.
5. Radovic-Moreno, A. F.; Chernyak, N.; Mader, C. C.; Subbarao, N.; Kang, R. S.; Hao, L.; Walker, D. A.; Halo, T. L.; Merkel, T. J.; Rische, C. H.; Anantatmula, S.; Burkhart, M.; Mirkin, C. A.; Gryaznov, S. M. (2015) Immunomodulatory spherical nucleic acids. Proceedings of the National Academy of Sciences, 112 (13) 3892-3897.
6. Pavlova, A. S.; Dovydenko, I. S.; Kupryushkin, M. S.; Grigor'eva, A. E.; Pyshnaya, I. A.; Pyshnyi, D. V. 2020. Amphiphilic “Like-A-Brush” Oligonucleotide Conjugates with Three Dodecyl Chains: Self-Assembly Features of Novel Scaffold Compounds for Nucleic Acids Delivery. Nanomaterials, 10(10), 1948.
7. Bousmail, D.; Amrein, L.; Fakhoury, J. J.; Fakih, H. H.; Hsu, J. C. C.; Panasci, L.; Sleiman, H. F. (2017) Precision spherical nucleic acids for delivery of anticancer drugs. Chemical Science, 8, 6218-6219.
8. Lv, Y.; Ruan, Z.; Wang, L.; Ni, B.; Wu, Y. (2009) Identification of a novel conserved HAL-A*0201-restricted epitope from the spike protein of SARS-CoV. BMC Immunology, 10(61).
9. U.S. Pat. No. 10,370,661
10. WO/2008/151049
11. U.S. Patent Application Publication No. 2020/0384104
12. WO/2019/118883
13. WO/2018/152327
14. U.S. Patent No. 10,182,988
15. WO/2015/126502
16. U.S. Patent Application Publication No. 2019/0060324
17. Radovic-Moreno AF, et al. PNAS. 2015; 112(13):3892-7.
18. Wang S, et al. PNAS. 2019; 116(21):10473-81. 3.
19. Clinicaltrials.gov Identifiers: NCT03684785, NCT03086278, NCT03020017

What is claimed is:

1. A spherical nucleic acid (SNA) comprising:
(a) a nanoparticle core; and
(b) a shell of oligonucleotides attached to the nanoparticle core, wherein one or more oligonucleotides in the shell of oligonucleotides is attached to the nanoparticle core through a hydrophobic anchor comprising one or more dodecane (C12) subunits.

2. The SNA of claim 1, wherein the hydrophobic anchor comprises 1 to about 20 dodecane subunits.

3. The SNA of claim 1 or claim 2, wherein the hydrophobic anchor comprises 9 dodecane subunits.

4. The SNA of any one of claims 1-3, wherein the SNA comprises an antigen, wherein the antigen is encapsulated in the nanoparticle core, attached to one or more oligonucleotides in the shell of oligonucleotides through a linker, attached to the external surface of the nanoparticle core through a linker, or a combination thereof.

5. The SNA of claim 4, wherein the antigen is attached through the linker to each oligonucleotide in the shell of oligonucleotides.

6. The SNA of claim 4 or claim 5, wherein the antigen is attached through the linker to an oligonucleotide that is hybridized to an oligonucleotide in the shell of oligonucleotides that is attached to the nanoparticle core.

7. The SNA of any one of claims 1-6, wherein the shell of oligonucleotides comprises an immunostimulatory oligonucleotide, a targeting oligonucleotide, an inhibitory oligonucleotide, a non-targeting oligonucleotide, or a combination thereof.

8. The SNA of claim 7, wherein the immunostimulatory oligonucleotide is a toll-like receptor (TLR) agonist.

9. The SNA of any one of claims 1-8, wherein each oligonucleotide in the shell of oligonucleotides is a toll-like receptor (TLR) agonist.

10. The SNA of claim 8 or claim 9, wherein the TLR is toll-like receptor 1 (TLR1), toll-like receptor 2 (TLR2), toll-like receptor 3 (TLR3), toll-like receptor 4 (TLR4), toll-like receptor 5 (TLRS), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), toll-like receptor 13 (TLR13), or a combination thereof.

11. The SNA of any one of claims 8-10, wherein the TLR is TLR9.

12. The SNA of any one of claims 7-11, wherein the immunostimulatory oligonucleotide comprises a CpG nucleotide sequence.

13. The SNA of any one of claims 1-12, wherein one or more oligonucleotides in the shell of oligonucleotides comprises or consists of the sequence of 5′-TCCATGACGTTCCTGACGTT-3′ (SEQ ID NO: 3).

14. The SNA of any one of claims 1-35, wherein one or more oligonucleotides in the shell of oligonucleotides comprises or consists of the sequence of 5′-TCGTCGTTTTGTCGTTTTGTCGTT-3′ (SEQ ID NO: 4).

15. The SNA of any one of claims 7-14, wherein at least about 10%, 20%, 30%, 40%, 50%, 60%, 70% 80%, 90%, 95%, or 100% of oligonucleotides in the shell of oligonucleotides is an immunostimulatory oligonucleotide.

16. The SNA of any one of claims 4-15, wherein the linker is a carbamate alkylene disulfide linker, a thiol linker, a disulfide linker, an amide alkylene disulfide linker, an amide alkylene thio-succinimidyl linker, or a combination thereof.

17. The SNA of any one of claims 1-16, wherein the nanoparticle core is a micellar core, a dendrimer core, a liposomal core, a lipid nanoparticle core, or a combination thereof.

18. The SNA of any one of claims 1-17, wherein the nanoparticle core is a liposome.

19. The SNA of claim 18, wherein the liposome comprises 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cholesterol, or a combination thereof.

20. The SNA of claim 18 or claim 19, wherein the liposome comprises 1,2-dipalmitoyl-sn-glycero-3-phosphocholine (DPPC).

21. The SNA of any one of claims 1-20, wherein each oligonucleotide in the shell of oligonucleotides comprises the hydrophobic anchor.

22. The SNA of any one of claims 1-21, wherein the hydrophobic anchor is attached to the 5′ end or the 3′ end of the one or more oligonucleotides.

23. The SNA of any one of claims 1-22, wherein the shell of oligonucleotides comprises DNA oligonucleotides, RNA oligonucleotides, or a combination thereof.

24. The SNA of any one of claims 1-23, wherein the shell of oligonucleotides comprises DNA oligonucleotides and RNA oligonucleotides.

25. The SNA of any one of claims 1-24, wherein the shell of oligonucleotides comprises single-stranded DNA, double-stranded DNA, single-stranded RNA, double-stranded RNA, or a combination thereof.

26. The SNA of any one of claims 1-25, wherein one or more oligonucleotides in the shell of oligonucleotides is a modified oligonucleotide.

27. The SNA of any one of claims 1-26, wherein the shell of oligonucleotides comprises about 2 to about 500 oligonucleotides.

28. The SNA of any one of claims 1-27, wherein the shell of oligonucleotides comprises about 75 oligonucleotides.

29. The SNA of any one of claims 1-28, wherein each oligonucleotide in the shell of oligonucleotides is about 5 to about 1000 nucleotides in length.

30. The SNA of claim 29, wherein each oligonucleotide in the shell of oligonucleotides is about 10 to about 50 nucleotides in length.

31. The SNA of any one of claims 1-30, wherein diameter of the SNA is about 1 nanometer (nm) to about 500 nm.

32. The SNA of any one of claims 1-31, wherein diameter of the SNA is less than or equal to about 80 nanometers.

33. The SNA of any one of claims 1-32, wherein diameter of the SNA is less than or equal to about 50 nanometers.

34. The SNA of any one of claims 7-33, wherein the inhibitory oligonucleotide is an antisense oligonucleotide, small interfering RNA (siRNA), an aptamer, a short hairpin RNA (shRNA), a DNAzyme, or an aptazyme.

35. The SNA of any one of claims 4-34, wherein the antigen is a tumor antigen, a viral antigen, a bacterial antigen, or a combination thereof.

36. A composition comprising a plurality of the SNA of any one of claims 1-35.

37. The composition of claim 36, wherein at least two SNAs in the plurality comprise a different nanoparticle core.

38. A pharmaceutical formulation comprising a plurality of the SNA of any one of claims 1-35, or the composition of claim 36 or claim 37, and a pharmaceutically acceptable carrier or diluent.

39. A vaccine comprising the SNA of any one of claims 1-35, the composition of claim 36 or claim 37, or the pharmaceutical formulation of claim 38.

40. The vaccine of claim 39, comprising an adjuvant.

41. An antigenic composition comprising the SNA of any one of claims 1-35 in a pharmaceutically acceptable carrier, diluent, stabilizer, or preservative, or the pharmaceutical formulation of claim 38, wherein the antigenic composition is capable of generating an immune response including dendritic cell activation, antibody generation, cytotoxic T cell activation, helper T cell activation, or a protective immune response in a subject.

42. The antigenic composition of claim 41, wherein the immune response includes an antibody response.

43. The antigenic composition of claim 42, wherein the antibody response is a neutralizing antibody response or a protective antibody response.

44. A method of inhibiting expression of a gene product comprising hybridizing a polynucleotide encoding the gene product to the inhibitory oligonucleotide of any one of claims 7-35, wherein hybridizing between the polynucleotide and the inhibitory oligonucleotide occurs over a length of the polynucleotide with a degree of complementarity sufficient to inhibit expression of the gene product.

45. The method of claim 44 wherein expression of the gene product is inhibited in vivo or in vitro.

46. A method of producing an immune response in a subject, comprising administering to the subject an effective amount of the SNA of any one of claims 1-35, the composition of claim 36 or claim 37, the pharmaceutical formulation of claim 38, the vaccine of claim 39 or claim 40, or the antigenic composition of any one of claims 41-43, thereby producing an immune response in the subject.

47. The method of claim 46, wherein the immune response includes an antibody response.

48. The method of claim 47, wherein the antibody response is a total antigen-specific antibody response.

49. The method of claim 47, wherein the antibody response is a neutralizing antibody response or a protective antibody response.

50. A method of immunizing a subject against one or more antigens comprising administering to the subject an effective amount of the SNA of any one of claims 1-35, the composition of claim 36 or claim 37, the pharmaceutical formulation of claim 38, the vaccine of claim 39 or 40, or the antigenic composition of any one of claims 41-43, thereby immunizing the subject against the one or more antigens.

51. The method of claim 50, wherein the composition or the vaccine is a cancer vaccine.

52. A method of treating a disorder comprising administering to a subject an effective amount of the SNA of any one of claims 1-35, the composition of claim 36 or claim 37, the pharmaceutical formulation of claim 38, the vaccine of claim 39 or 40, or the antigenic composition of any one of claims 41-43, thereby treating the disorder in the subject.

53. The method of claim 52, wherein the disorder is a cancer or an infection.

54. The method of claim 53, wherein the infection is a viral infection or a bacterial infection.

55. The method of claim 51 or claim 53, wherein the cancer is bladder cancer, breast cancer, cervical cancer, colon cancer, rectal cancer, endometrial cancer, glioblastoma, kidney cancer, leukemia, liver cancer, lung cancer, melanoma, lymphoma, non-Hodgkin lymphoma, osteocarcinoma, ovarian cancer, pancreatic cancer, prostate cancer, thyroid cancer, and human papilloma virus-induced cancer, or a combination thereof.

56. The method of any one of claims 46-55, further comprising administering a therapeutic agent.

57. The method of claim 56, wherein the therapeutic agent is an anti-programmed cell death protein 1 (PD-1) antibody, an anti-programmed death-ligand 1 (PD-L1) antibody, a cytotoxic T lymphocyte antigen 4 (CTLA-4) antibody, or a combination thereof.

58. The method of any one of claims 44-57, wherein the SNA is the SNA of claim 20.

59. The method of any one of claims 44-57, wherein the SNA is the SNA of claim 28.

60. The method of any one of claims 46-59, wherein the administering results in a less than 25% increase in serum level of a cytokine in the subject compared to serum level of the cytokine in a subject that was not administered the SNA.

61. The method of claim 60, wherein the cytokine is interleukin-2 (IL-2), interleukin-6 (IL-6), interleukin-10 (IL-10), C-X-C Motif Chemokine Ligand 1 (CXCL1), tumor necrosis factor α (TNF-α), interleukin-1 (IL-1), interferon, or a combination thereof.


SEQUENCE LISTING (4 sequences)

SEQ ID NO: 1 - protein, 9 aa, Artificial Sequence
  Synthetic
VLNDILSRL

SEQ ID NO: 2 - protein, 9 aa, Artificial Sequence
  Synthetic
SAVKNFTEI

SEQ ID NO: 3 - DNA, 20 bp, Artificial Sequence
  Synthetic
tccatgacgttcctgacgtt

SEQ ID NO: 4 - DNA, 24 bp, Artificial Sequence
  Synthetic
tcgtcgttttgtcgttttgtcgtt